(19) United States
(12) Patent Application Publication
(10) Pub. No.: US 2023/0232763 A1
(43) Pub. Date: Jul. 27, 2023

(54) SOYBEAN WITH ALTERED SEED PROTEIN

(71) Applicant: PIONEER HI-BRED INTERNATIONAL, INC., JOHNSTON, IA (US)

(72) Inventors: ZHENGLIN HOU, ANKENY, IA (US); ZHAN-BIN LIU, CLIVE, IA (US); KRISHNA M. MADDURI, CARMEL, IN (US); BO SHEN, JOHNSTON, IA (US); JUN ZHANG, JOHNSTON, IA (US)

(73) Assignee: PIONEER HI-BRED INTERNATIONAL, INC., JOHNSTON, IA (US)

(21) Appl. No.: 17/996,639

(22) PCT Filed: Apr. 20, 2021
(86) PCT No.: PCT/US2021/028058

§ 371 Date: Oct. 20, 2022

Related U.S. Application Data
(60) Provisional application No. 63/014,316, filed on Apr. 23, 2020.

(51) Int. Cl. A01H 5/10; A01H 6/54

(52) U.S. Cl. CPC A01H 5/10; A01H 6/542

(57) ABSTRACT

Soybean plants and soybean seeds comprise increased amounts of essential amino acids such as methionine, tryptophan or lysine. Soybean plants, soybean seeds and soy protein compositions comprising a modified glycinin protein, modified amounts of glycinin and beta-conglycinin, or both have increases in one or more essential amino acids. Protein compositions having increased essential amino acids can be made from the soybean seeds. Additionally, methods for generating and using plants, seeds and protein compositions having increased amounts of essential amino acids are disclosed.

### REFERENCE TO SEQUENCE LISTING SUBMITTED ELECTRONICALLY

The official copy of the sequence listing is submitted electronically via EFS-Web as an ASCII formatted sequence listing with a file named 8427-US-PSP_SequenceListing_ST25.txt created on 17 Apr. 2020 and having a size of 140 kilobytes and is filed concurrently with the specification. The sequence listing comprised in this ASCII formatted document is part of the specification and is herein incorporated by reference in its entirety.

### FIELD OF THE INVENTION

This disclosure relates to the field of molecular biology.

### BACKGROUND

Among the 20 amino acids, animals cannot synthesize the nine essential amino acids and, as a result, these essential amino acids, Lys, Met, Thr, Phe, Try, Val, Ile, Leu and His, must be acquired from food. In soybeans, certain essential amino acids limit the nutritional quality of the meal because their content is insufficient to meet the optimal growth needs of animals.

Compositions and methods to enhance the nutritional quality of soybeans by increasing the amount of essential amino acids are provided.

### SUMMARY OF INVENTION

Provided herein are soybean plants or soybean seeds comprising a polynucleotide encoding a modified glycinin protein having an amino acid sequence that is at least 80% identical to SEQ ID NO: 1 and having at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, and 486MMMMMM487, or any combination thereof. In certain embodiments, the soybean plant or soybean seed further comprises at least one additional modification selected from the group consisting of a modification decreasing the expression of beta-conglycinin, a modification increasing the expression of a polynucleotide encoding cystathionine-gamma-synthase (CGS), a modification decreasing the expression and/or activity of MGL, a modification decreasing the expression and/or activity of LKR/SDH, and a modification increasing the activity of DHPS, or any combination thereof. In certain embodiments, the one or more modifications is a modification of the endogenous gene (e.g., endogenous glycinin). In certain embodiments, the modified glycinin protein comprises at least a 25% increase in the amount of methionine, tryptophan, or methionine and tryptophan as compared to SEQ ID NO: 1.

Also provided herein are soybean seeds or soybean plants producing soybean seeds comprising at least about a 10% increase in one or more essential amino acids, as compared to a control seed, wherein the soybean seed or soybean plant comprises at least one modification selected from the group consisting of a targeted genetic modification of a gene encoding a glycinin protein to produce a modified glycinin protein comprising an insertion or substitution of one or more methionine, threonine, tryptophan, or lysine residues, or any combination thereof, a modification decreasing the expression of beta-conglycinin, a modification increasing the expression of a polynucleotide encoding CGS, a modification decreasing the expression and/or activity of MGL, a modification decreasing the expression and/or activity of LKR/SDH, or a modification increasing the activity of DHPS. In certain embodiments, the modified glycinin protein comprises an amino acid sequence that is at least 80% identical to SEQ ID NO: 1. In certain embodiments the modified glycinin protein comprises an amino acid sequence that is at least 80% identical to SEQ ID NO: 1 and comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof. In certain embodiments, the modified glycinin protein comprises at least a 40% increase in the amount of methionine, tryptophan, methionine and tryptophan, threonine, and/or lysine as compared to SEQ ID NO: 1. In certain embodiments, the seed produced from the soybean plant or the soybean seed comprises at least a 15% increase in the amount of methionine, tryptophan, methionine and tryptophan, threonine, and/or lysine as compared to a control seed.

Also provided herein is a soybean seed or a soybean plant producing a soybean seed comprising at least about a 20% increase in methionine on a dry weight basis and at least a 2% point increase in total protein measured on a dry weight basis, as compared to a control seed. In certain embodiments, the soybean seed or soybean plant comprises a modification selected from the group consisting of (i) a targeted genetic modification of a gene encoding a glycinin protein to produce a modified glycinin protein comprising an insertion or substitution of one or more methionine, threonine, tryptophan, or lysine residues, or any combination thereof, (ii) a modification decreasing the expression of beta-conglycinin, (iii) a modification increasing the expression of a polynucleotide encoding cystathionine-gamma-synthase (CGS), (iv) a modification decreasing the expression and/or activity of methionine beta-lyase (MGL), (v) a modification decreasing the expression and/or activity of LKR/SDH, (vi) a modification increasing the expression or activity of dihydrodipicolinate synthase (DHPS), and (vii) any combination thereof. In certain embodiments, the modified glycinin protein comprises an amino acid sequence that is at least 80% identical to SEQ ID NO: 1. In certain embodiments the modified glycinin protein comprises an amino acid sequence that is at least 80% identical to SEQ ID NO: 1 and comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof. In certain embodiments, the modified glycinin protein comprises at least a 40% increase in the amount of methionine, tryptophan, methionine and tryptophan, threonine, and/or lysine as compared to SEQ ID NO: 1. In certain embodiments, the seed produced from the soybean plant or the soybean seed comprises at least a 15% increase in the amount of methionine, tryptophan, methionine and tryptophan, threonine, and/or lysine as compared to a control seed.

Also provided herein is a method of producing a soybean plant having an increased seed essential amino acid content as compared to a control seed comprising introducing into a regenerable plant cell at least one targeted genetic modification selected from the group consisting of a targeted genetic modification of a gene encoding a glycinin protein to produce a modified glycinin protein comprising an insertion or substitution of one or more methionine, threonine, tryptophan, or lysine residues, or any combination thereof, a modification decreasing the expression of beta-conglycinin, a modification increasing the expression of a polynucleotide encoding CGS, a modification decreasing the expression and/or activity of MGL, a modification decreasing the expression and/or activity of LKR/SDH, or a modification increasing the activity of DHPS; and generating the plant, wherein the plant comprises the at least one targeted genetic modification. In certain embodiments the modified glycinin protein comprises an amino acid sequence that is at least 80% identical to SEQ ID NO: 1 and comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof. In certain embodiments, the method produces a seed comprising at least a 30% increase in the amount of methionine, tryptophan, methionine and tryptophan, threonine, and/or lysine as compared to a control seed. In certain embodiments, the at least one targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases. In certain embodiments, the targeted genetic modification is present (a) in the coding region; (b) a non-coding region; (c) a regulatory sequence; (d) an untranslated region; or (e) any combination of (a)-(d). In certain embodiments, the method further comprises producing a soy protein composition produced from the plant generated.

Further provide herein is a soy protein composition having an essential amino acid content of at least 50% of the total amino acid and at least one characteristic selected from the group consisting of: (a) a modified glycinin protein comprising at least a 10% increase in the proportion of methionine residues, tryptophan residues or a combination thereof of the total glycinin amino acid residues compared with a control glycinin protein; (b) the composition comprises less than 5% beta-conglycinin; (c) a CGS protein comprising an amino acid sequence that is at least 75% identical to any one of SEQ ID NOs: 35-37; (d) the composition lacks MGL; (e) the composition lacks LKR/SDH; (f) a modified DHPS protein, and (g) the composition comprises less than about 5% RFO. In certain embodiments the modified glycinin protein comprises an amino acid sequence that is at least 80% identical to SEQ ID NO: 1 and comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof. In certain embodiments, the sum of the methionine and tryptophan in the soy protein composition is greater than about 20, 30, 40, 50, 60, 70, 75, 80, 80, 100, 110, 120, 130, 140 or 150 mg/g protein and less than 250, 200, 150, 100, 90, 80, 75, 70 or 60 mg/g protein. In certain embodiments, the sum of the methionine, lysine, threonine and tryptophan in the soy protein composition is greater than about 50, 60, 70, 75, 80, 90, 100, 110, 120, 130, 140, 150, 160, 170, 180 or 200 mg/g protein and less than 300, 250, 200, 150, 100, 90, 80 or 75 mg/g protein. In certain embodiments, the soy protein composition comprises the at least one genetic modification and has an essential amino acid content of at least 50%.

In certain embodiments, the soybeans and methods disclosed herein can be used to produce soy protein products such as whole soybean products as for example roasted soybeans, baked soybeans, soy sprouts, and soy milk, or to produce processed soy protein products such as full fat and defatted flours, soy grits, soy hypocotyls, soybean meal, soy milk, soy milk powder, soy protein isolates or with specialty soy foods and ingredients, such as soy milk, tofu, tempeh, miso, soy sauce, hydrolyzed vegetable protein and whipping protein, or to produce soy protein concentrates, textured soy proteins, textured flours and concentrates, textured concentrates, textured isolates, and soy crisps to improve for example wettability and nutritional composition for food and feed applications. Soy protein concentrates refer to those products produced from dehulled, defatted soybeans and typically contain 65 wt% to 90 wt% soy protein on a moisture free basis. As used herein, the term “soy protein isolate” or “isolated soy protein” refers to a soy protein containing material that contains at least 90% soy protein by weight on a moisture free basis. Soy protein products produced from soybeans described herein can be incorporated into food, beverages, and animal feed.

In certain embodiments, the soy protein composition is an animal feed or human food, such as, for example, a) soybean meal; b) soyflour; c) defatted soyflour; d) soymilk; e) spray-dried soymilk; f) soy protein concentrate; g) texturized soy protein concentrate; h) hydrolyzed soy protein; i) soy protein isolate; j) spray-dried tofu; k) soy meat analog; 1) soy cheese analog; and m) soy coffee creamer. Processing steps for soybean seeds disclosed herein to produce the protein compositions described herein include one or more of dehulling of the soybeans, extraction of oil, for example by use of solvents such as hexane, processing soy flakes to soy meal for animal feed, grinding soy flakes to produce soy flour, sizing soy flakes to produce soy grits or texturizing soy flakes to produce textured vegetable protein. Soy protein concentrates and isolated soy protein can be further refined and produced from soy flakes.

In certain embodiments of the soybean plants, soybean seeds, and soy protein compositions, the at least one amino acid modification of the modified glycinin protein comprises (a) P300WWWWWWW; (b) Q196M, Q197M, E198M, Q199M, and Q203M; (c) 486MMMMMM487; (d) Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, and 486MMMMMM487; (e) L39M, L51M, L74M, L141M, L175M, L193M, L226M, L262M, L321M, L347M, L376M, L385M, and L390M; (f) L39M, L51M, L74M, L141M, L175M, L193M, L226M, L262M, L321M, L347M, L376M, L385M, L390M, Y104W, Y134W, Y153W, Y334W, F361W, Y431W, and F480W; or (g) Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, 486MMMMMM487, Y104W, Y134W, Y153W, Y334W, F361W, Y431W, and F480W. In certain embodiments of the soybean plants, soybean seeds, and soy protein compositions, the modified glycinin protein comprises an amino acid sequence that is at least 80% identical to any one of SEQ ID NOs: 4-12, wherein the sequence comprises at least one modification described herein.

In certain embodiments of the soybean plants, soybean seeds, and soy protein compositions, the modification decreasing the expression of beta-conglycinin comprises a knockout of one or more beta-conglycinin genes encoding one or more isoforms of beta-conglycinin comprising an amino acid sequence that is at least 90% identical to any one of SEQ ID NOs: 57-63. In certain embodiments of the soybean plants, soybean seeds, and soy protein compositions, the modification decreasing the expression of beta-conglycinin comprises a knockout of two or more beta-conglycinin genes encoding at least 5 isoforms of beta-glycinin comprising an amino acid sequence that is at least 90% identical to any one of SEQ ID NOs: 57-63. In certain embodiments of the soybean plants, soybean seeds, and soy protein compositions, the ratio of glycinin to conglycinin in the soybean plant or soybean seed is at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 65, 70, 75, 80, 85, 90, 95, 100, 500 or 1000 to 1.

In certain embodiments of the soybean plants, soybean seeds, and soy protein compositions, the modification increasing the expression of a polynucleotide encoding CGS comprises a targeted genetic modification that removes a self-regulatory domain of a CGS gene, wherein the self-regulatory domain of the CGS gene encodes a polypeptide comprising an amino acid sequence that is at least 90% identical to SEQ ID NO: 64. In certain embodiments of the soybean plants, soybean seeds, and soy protein compositions, the modified CGS gene encodes a CGS protein comprising an amino acid sequence that is at least 90% identical to any one of SEQ ID NOs: 35-37.

In certain embodiments of the soybean plants, soybean seeds, and soy protein compositions, the modification decreasing the expression and/or activity of MGL comprises a knockout of the MGL gene, wherein the MGL gene encodes an MGL protein comprising an amino acid sequence that is at least 90% identical to SEQ ID NO: 53 or 54.

In certain embodiments of the soybea plants, soybean seeds, and soy protein compositions, the expression and/or activity of LKR/SDH comprises a knockout of the LKR/SDH gene, wherein the LKR/SDH gene encodes a LKR/SDH protein comprising an amino acid sequence that is at least 90% identical to SEQ ID NO: 55 or 56.

In certain embodiments, the soybean plants or soybean seeds further comprise at least one modification increasing the total protein in the seed as compared to a control seed (e.g., seed not comprising the at least one modification). In certain embodiments, the soybean seed comprising the at least one modification comprises at least about a 2% percentage point increase in total protein measured on a dry weight basis, as compared to a control seed.

In certain embodiments, the soybean plants or soybean seeds further comprise at least one modification decreasing the raffinose family oligosaccharides (RFO) content in the seed. In certain embodiments, the modification comprises a decrease in the expression and/or activity of a raffinose synthase. In certain embodiments, the modification comprises a decrease in the expression and/or activity of raffinose synthase 2 (RS2) and/or raffinose synthase 4 (RS4). In certain embodiments, the soybean seed comprises at least a 35%, 40%, 45%, 50%, 55%, 60%, 65%, 70%, 75%, 80%, 85%, 90%, 95%, or 99% decrease in expression of RS2, RS4, or RS2 and RS4, as compared to a control seed. In certain embodiments, the seed comprises less than about 6%, 5.5%, 5%, 4.5%, 4%, 3.5%, 3%, 2.5%, 2%, 1.5%, 1%, or 0.5% RFO content on a dry weight basis. In certain embodiments, the introduced modification decreases RFO content by at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

In certain embodiments of the soybean plants, soybean seeds, and soy protein compositions, the expression and/or activity of LKR/SDH comprises a knockout of the LKR/SDH gene, wherein the LKR/SDH gene encodes an LKR/SDH protein comprising an amino acid sequence that is at least 90% identical to SEQ ID NO: 55 or 56.

Further provided is a method of plant breeding comprising crossing any one of the soybean plants provided herein with a second soybean plant to produce a progeny seed.

Also provided are plants produced from the seed described herein, wherein the plant produced comprises at least one modification described herein.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 provides experimental results from a Western blot showing the detection of pro-glycinin1 and its basic subunit using anti-glycinin1 antibody from total protein extract of soybean seeds treated with (+) or without (-) 1,4-Dithiothreitol (DTT). Star shows the native proglycinin at 53.6 kDa and arrowhead indicates the basic subunit.

FIG. 2 provides experimental results from a Western blot analysis of wide-type and modified glycinin1 proteins that were expressed in the BY-2 cell free system. The arrow indicates the proglycinin1 bands at the expected molecular weight after the signal peptide was removed in microsomes. Protein extract from control soybean seeds treated with DTT (+DTT) or without DTT (-DTT) shows the native proglycinin1 (star) and the basic subunit (arrowhead). MK is mock transfected, WT is wild type glycinin, and the glycinin variants correspond to SEQ ID NOs: 9 (V8); 4 (V9); 10 (V10); 5 (V11); 6 (V12); 7 (V13); 8 (V14); and 11 (V15)

FIG. 3 provides a partial sequence alignment SEQ ID NO: 33 and 35 depicting an editing variant created for the CGS1 and a partial sequence alignment of SEQ ID NOs: 34, 36 and 37 depicting editing variants created for the CGS2 gene.

FIG. 4 provides a schematic of the isoforms of beta-conglycinin and the gRNAs used to delete 6 of the 7 beta-conglycinin isoforms.

FIG. 5 provides experimental data showing the knockout of beta-conglycinin alpha’ subunits in homozygous T2 dropout seeds as compared to a wild-type (WT) control seed. No alpha’ subunits of conglycinin proteins can be detected in the T2 homozygous seeds from the dropout variants, demonstrating complete removal of the conglycinin alpha’ subunit proteins in soybean seeds.

FIG. 6 provides experimental data showing the knockout of beta-conglycinin alpha subunits in homozygous (Hom) or heterozygous (Het) T2 dropout seeds as compared to a wild-type (WT) control seed.

The sequence descriptions (Table 1) summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1

Sequence Listing Description

| SEQ ID NO: | Name | Mutation |
|---|---|---|
| 1 | Glycinin1 Amino Acid | |
| 2 | Glycinin1 Nucleotide | |
| 3 | Peptide used for Glycinin 1 antibody | |
| 4 | Glycinin_V9 Amino Acid | P300WWWWWWW |
| 5 | Glycinin _V11 Amino Acid | Q118MMM Q119MMM |
| 6 | Glycinin_V12 Amino Acid | Q196M Q197M E198M Q199M Q203M |
| 7 | Glycinin_V13 Amino Acid | 486MMMMMM487 |
| 8 | Glycinin_V14 Amino Acid | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 |
| 9 | Glycinin_V8 Amino Acid | L39M L51M L74M L141M L175M L193M L226M L262M L321M L347M L376M L385M L390M |
| 10 | Glycinin_V10 Amino Acid | L39M L51M L74M L141M L175M L193M L226M L262M L321M L347M L376M L385M L390M & Y104W Y134W Y153W Y334W F361W Y431W F480W |
| 11 | Glycinin_V15 Amino Acid | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 & Y104W Y134W Y153W Y334W F361W Y431W F480W |
| 12 | Glycinin_V16 Amino Acid | L39M L51M L74M Q 118MMM Q119MMMY104WY134W L141M Y153W L175M L193M Q196M Q197M E198M Q199M Q203M L226M L262M P300MMMMMMM L321M Y334W L347M F361W L376M L385M L390M Y431W F480W 486MMMMMM487 |
| 13 | Glycinin_V9 Nucleotide | P300WWWWWWW |
| 14 | Glycinin_V11 Nucleotide | Q118MMM Q119MMM |
| 15 | Glycinin_V12 Nucleotide | Q196M Q197M E198M Q199M Q203M |
| 16 | Glycinin_V13 Nucleotide | 486MMMMMM487 |
| 17 | Glycinin_V14 Nucleotide | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 |
| 18 | Glycinin_V8 Nucleotide | L39M L51M L74M L141M L175M L193M L226M L262M L321M L347M L376M L385M L390M |
| 19 | Glycinin_V10 Nucleotide | L39M L51M L74M L141M L175M L193M L226M L262M L321M L347M L376M L385M L390M & Y104W Y134W Y153W Y334W F361W Y431W F480W |
| 20 | Glycinin_V15 Nucleotide | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 & Y104W Y134W Y153W Y334W F361W Y431W F480W |
| 21 | Glycinin_V16 Nucleotide | L39M L51M L74M Q 118MMM Q119MMMY104WY134W L141M Y153W L175M L193M Q196M Q197M E198M Q199M |
| | | Q203M L226M L262M P300MMMMMMM L321M Y334W L347M F361W L376M L385M L390M Y431W F480W 486MMMMMM487 |
| 22 | GM-CGS-gRNA1 | |
| 23 | GM-CGS-gRNA2 | |
| 24 | GM-CGS-gRNA3 | |
| 25 | GM-CONG-gRNA1 | |
| 26 | GM-CONG-gRNA2 | |
| 27 | GM-CONG-gRNA3 | |
| 28 | GM-CONG-gRNA4 | |
| 29 | GM-deCGS | |
| 30 | GM-βCon_RNAi | |
| 31 | GM-CGS1 Exon1 CDS | |
| 32 | GM-CGS2 Exon1 CDS | |
| 33 | GM-CGS1 Exon1 WT | |
| 34 | GM-CGS2 Exon1 WT | |
| 35 | GM-CGS1 261nt in-frame deletion Exon1 edited variant 1 | |
| 36 | GM-CGS2 261nt in-frame deletion Exon1 edited variant 1 | |
| 37 | GM-CGS2 276nt in-frame deletion Exon1 edited variant2 | |
| 38 | GM-CGS1-gRNA1 | |
| 39 | GM-CGS2-gRNA1 | |
| 40 | GM-CONG-gRNA5 | |
| 41 | GM-CONG-gRNA6 | |
| 42 | GM-CONG-gRNA7 | |
| 43 | GM-CGS1 wild-type Amino Acid | |
| 44 | GM-CGS2 wild-type Amino Acid | |
| 45 | GM-MGL1 Exon1 CDS | |
| 46 | GM-MGL2 Exon1 CDS | |
| 47 | GM-MGL-gRNA1 | |
| 48 | GM-MGL-gRNA2 | |
| 49 | GM-LKR-gRNA1 | |
| 50 | GM-LKR-gRNA2 | |
| 51 | GM-LKR-gRNA3 | |
| 52 | GM-LKR-gRNA4 | |
| 53 | Methionine beta-lyase (MGL) Amino Acid 1 | |
| 54 | Methionine beta-lyase (MGL) Amino Acid 2 | |
| 55 | Lysine-ketoglutarate reductase /Saccharopine dehydrogenase (LKR/SDH) Amino Acid 1 | |
| 56 | Lysine-ketoglutarate reductase /Saccharopine dehydrogenase (LKR/SDH) Amino Acid 2 | |
| 57 | Beta-conglycinin isoform Glyma.20g148200 (β) | |
| 58 | Beta-conglycinin isoform Glyma.20g148300 (α) | |
| 59 | Beta-conglycinin isoform Glyma.20g148400 (α) | |
| 60 | Beta-conglycinin isoform Glyma.20g146200 (β) | |
| 61 | Beta-conglycinin isoform Glyma.10g246300 (α′) | |
| 62 | Beta-conglycinin isoform Glyma.10g246500 (α′) | |
| 63 | Beta-conglycinin isoform Glyma.10g246400 (α) | |
| 64 | CGS1 self-regulatory domain | |
| 65 | GM-RS2-gRNA1 | |
| 66 | GM-RS2-gRNA2 | |
| 67 | GM-RS4-gRNA1 | |
| 68 | GM-RS4-gRNA2 | |
| 69 | GM-RS2-gRNA3 | |
| 70 | GM-RS3-gRNA1 | |
| 71 | GM-RS4-gRNA3 | |

### DETAILED DESCRIPTION

### I. Compositions

### A. Glycinin Polynucleotides and Polypeptides

The present disclosure provides modified glycinin polynucleotides and polypeptides. Soybean glycinin, a member of 11S globulin family, forms a hexamer and each subunit is composed of an acidic (35 kDa) and a basic (20 kDa) polypeptide, which are linked together by disulfide bonds.

Provided are glycinin polynucleotides encoding polypeptides having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1 and a modification described herein. Provided are glycinin polynucleotides having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO:2 and encoding a modification described herein.

One aspect of the disclosure provides a polynucleotide encoding a modified glycinin polypeptide comprising an amino acid sequence that is at least 50% (e.g., at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) identical to SEQ ID NO: 1 and comprises at least one amino acid substitution or insertion selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, or 486MMMMMM487, or any combination thereof.

As should be understood by those of ordinary skill in the art, a mutation of, for example, Q196M of SEQ ID NO: 1 indicates a substitution mutation in which the glutamine (Q) at position 196 of SEQ ID NO: 1 is mutated to a methionine (M). Similarly, as should be understood by those of ordinary skill in the art, a mutation of, for example, P300WWWWWWW of SEQ ID NO: 1 indicates a substitution mutation in which the proline (P) at position 300 of SEQ ID NO: 1 is substituted with seven tryptophan residues, such that six additional tryptophan residues are inserted between the W at position 300 and the amino acid at position 301 of SEQ ID NO: 1.

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of methionine residues in the modified glycinin polypeptide as compared to the glycinin polypeptide of SEQ ID NO: 1.

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of tryptophan residues in the modified glycinin polypeptide as compared to the glycinin polypeptide of SEQ ID NO: 1.

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of both methionine and tryptophan residues in the modified glycinin polypeptide as compared to the glycinin polypeptide of SEQ ID NO: 1.

In certain embodiments, the modified glycinin polypeptide comprises an amino acid sequence that is at least 80% identical to SEQ ID NO: 1 and comprises, consists essentially of, or consists of P300WWWWWWW.

In certain embodiments, the modified glycinin polypeptide comprises an amino acid sequence that is at least 80% identical to SEQ ID NO: 1 and comprises, consists essentially of, or consists of Q196M, Q197M, E198M, Q199M, and Q203M.

In certain embodiments, the modified glycinin polypeptide comprises an amino acid sequence that is at least 80% identical to SEQ ID NO: 1 and comprises, consists essentially of, or consists of 486MMMMMM487.

In certain embodiments, the modified glycinin polypeptide comprises an amino acid sequence that is at least 80% identical to SEQ ID NO: 1 and comprises, consists essentially of, or consists of Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, and 486MMMMMM487.

In certain embodiments, the modified glycinin polypeptide comprises an amino acid sequence that is at least 80% identical to SEQ ID NO: 1 and comprises, consists essentially of, or consists of L39M, L51M, L74M, L141M, L175M, L193M, L226M, L262M, L321M, L347M, L376M, L385M, and L390M.

In certain embodiments, the modified glycinin polypeptide comprises an amino acid sequence that is at least 80% identical to SEQ ID NO: 1 and comprises, consists essentially of, or consists of L39M, L51M, L74M, L141M, L175M, L193M, L226M, L262M, L321M, L347M, L376M, L385M, L390M, Y104W, Y134W, Y153W, Y334W, F361W, Y431W, and F480W.

In certain embodiments, the modified glycinin polypeptide comprises an amino acid sequence that is at least 80% identical to SEQ ID NO: 1 and comprises, consists essentially of, or consists of Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, 486MMMMMM487, Y104W, Y134W, Y153W, Y334W, F361W, Y431W, and F480W.

In certain embodiments, the modified glycinin polypeptide comprises an amino acid sequence that is at least 80% identical to any one of SEQ ID NOs: 4-12. In certain embodiments, the modified glycinin polypeptide comprising an amino acid sequence that is at least 80% identical to any one of SEQ ID NOs: 4-12 and comprises at least one mutation described herein.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, a percent similarity may be used. Sequences, which differ by such conservative substitutions, are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, California, USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, “reference sequence” is a defined sequence used as a basis for sequence comparison. A reference sequence may be a subset or the entirety of a specified sequence, for example, as a segment of a full-length cDNA or gene sequence or the complete cDNA or gene sequence.

As used herein, “comparison window” means reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, California, GAP, BESTFIT, BLAST, FASTA and TFASTA in the Wisconsin Genetics Software Package®, Version 8 (available from Genetics Computer Group (GCG® programs (Accelrys, Inc., San Diego, CA)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

In certain embodiments, the modified glycinin polynucleotides described above are inserted into a recombinant DNA construct. In certain embodiments, the recombinant DNA construct further comprises at least one regulatory element. In certain embodiments, the at least one regulatory element of the recombinant DNA construct comprises a promoter, preferably a heterologous promoter.

As used herein, a “recombinant DNA construct” comprises two or more operably linked DNA segments which are not found operably linked in nature. Non-limiting examples of recombinant DNA constructs include a polynucleotide of interest operably linked to heterologous sequences, also referred to as “regulatory elements,” which aid in the expression, autologous replication, and/or genomic insertion of the sequence of interest. Such regulatory elements include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The modified glycinin described herein can be provided for expression in a plant of interest or an organism of interest. The cassette can include 5′ and 3′ regulatory sequences operably linked to a modified glycinin polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the modified glycinin polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a modified glycinin polynucleotide described herein, and a transcriptional and translational termination region (e.g., termination region) functional in plants. The regulatory regions (e.g., promoters, transcriptional regulatory regions, and translational termination regions) and/or the modified glycinin polynucleotide may be native/analogous to the host cell or to each other. Alternatively, the regulatory regions and/or the modified glycinin polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the modified glycinin polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last etal. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various modified glycinin sequences disclosed herein, including the native promoter of the polynucleotide sequence of interest. The promoters for use in the recombinant DNA constructs of the invention can be selected based on the desired outcome.

In certain embodiments, the recombinant DNA construct, described herein is expressed in a plant or seed. In certain embodiment, the plant or seed is a soybean plant or soybean seed. As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides. The polynucleotides or recombinant DNA constructs disclosed herein may be used for transformation of any plant species

### B. Soybean Plants or Soybean Seeds Comprising a Modified Glycinin Protein

The present disclosure further provides soybean plants or soybean seeds comprising a polynucleotide encoding a modified glycinin protein comprising an amino acid sequence that is at least 80% identical to SEQ ID NO: 1 and having at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, and 486MMMMMM487, or any combination thereof.

Soybean glycinin is a member of 11S globulin family and accounts for about one third of the total seed proteins. Glycinin protein forms a hexamer and each subunit is composed of an acidic (35 kDa) and a basic (20 kDa) polypeptide, which are linked together by disulfide bonds. Glycinin is synthesized first as a precursor (pre-pro-glycinin) before a signal peptide is removed in the endoplasmic reticulum. Pro-glycinin is then assembled into trimers and sorted to protein storage vacuoles (PSV). Further post-translational cleavage between Asn and Gly produces mature glycinin consisting of acidic and basic polypeptides, which are then assembled into a hexamer of about 11S in PSV (Utsumi et al., Food Science and Technology, 257-292 (1997)).

Glycinin1 (A1aB1b, Glyma.03G32030 or Glyma.03G163500.1, SEQ ID NO: 1) is one of the major isoforms of glycinin protein, and makes up about 11% of total soybean protein (Utsumi et al., Food Science and Technology, 257-292 (1997)).

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of methionine residues in the modified glycinin polypeptide as compared to the glycinin polypeptide of SEQ ID NO: 1.

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of tryptophan residues in the modified glycinin polypeptide as compared to the glycinin polypeptide of SEQ ID NO: 1.

In certain embodiments, the modified glycinin protein comprises at least a 25%, 30%, 40%, 50%, 60%, 70%, 80%, 90%, 100%, 150%, 200%, 300%, 400%, 500%, 600%, 700%, or 800% and less than a 1500%, 1400%, 1300%, 1200%, 1100%, 1000%, 900%, 800%, 700%, 600%, 500%, 400%, 300%, 200%, or 100% increase in the number of both methionine and tryptophan residues in the modified glycinin polypeptide as compared to the glycinin polypeptide of SEQ ID NO: 1.

In certain embodiments, the modified glycinin polypeptide comprises an amino acid sequence that is at least 80% identical to SEQ ID NO: 1 and comprises, consists essentially of, or consists of P300WWWWWWW.

In certain embodiments, the modified glycinin polypeptide comprises an amino acid sequence that is at least 80% identical to SEQ ID NO: 1 and comprises, consists essentially of, or consists of Q196M, Q197M, E198M, Q199M, and Q203M.

In certain embodiments, the modified glycinin polypeptide comprises an amino acid sequence that is at least 80% identical to SEQ ID NO: 1 and comprises, consists essentially of, or consists of 486MMMMMM487.

In certain embodiments, the modified glycinin polypeptide comprises an amino acid sequence that is at least 80% identical to SEQ ID NO: 1 and comprises, consists essentially of, or consists of Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, and 486MMMMMM487.

In certain embodiments, the modified glycinin polypeptide comprises an amino acid sequence that is at least 80% identical to SEQ ID NO: 1 and comprises, consists essentially of, or consists of L39M, L51M, L74M, L141M, L175M, L193M, L226M, L262M, L321M, L347M, L376M, L385M, and L390M.

In certain embodiments, the modified glycinin polypeptide comprises an amino acid sequence that is at least 80% identical to SEQ ID NO: 1 and comprises, consists essentially of, or consists of L39M, L51M, L74M, L141M, L175M, L193M, L226M, L262M, L321M, L347M, L376M, L385M, L390M, Y104W, Y134W, Y153W, Y334W, F361W, Y431W, and F480W.

In certain embodiments, the modified glycinin polypeptide comprises an amino acid sequence that is at least 80% identical to SEQ ID NO: 1 and comprises, consists essentially of, or consists of Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, 486MMMMMM487, Y104W, Y134W, Y153W, Y334W, F361W, Y431W, and F480W.

In certain embodiments, the modified glycinin polypeptide comprises an amino acid sequence that is at least 80% identical to any one of SEQ ID NOs: 4-12. In certain embodiments, the modified glycinin polypeptide comprising an amino acid sequence that is at least 80% identical to any one of SEQ ID NOs: 4-12 and comprises at least one mutation described herein.

In certain embodiments, an endogenous glycinin sequence is modified to encode the modified glycinin protein. In certain embodiments, the endogenous glycinin sequence is modified using a genome modification technique selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases.

In certain embodiments, the soybean plant or soybean seed further comprises at least one additional modification selected from the group consisting of a modification decreasing the expression of beta-conglycinin, a modification increasing the expression of a polynucleotide encoding at least one cystathionine-gamma-synthase (CGS) (e.g. CGS-1 and/or CGS-2), a modification decreasing the expression and/or activity of methionine beta-lyase (MGL), a modification decreasing the expression and/or activity of lysine-ketoglutarate reductase /Saccharopine dehydrogenase (LKR/SDH), a modification increasing the activity of dihydrodipicolinate synthase (DHPS), or any combination thereof.

In soybean seeds, β-conglycinin, the abundant 7S globulin storage protein, and glycinin consist of about 21% and 33% of total protein content, respectively (Utsumi et al., Food Science and Technology, 257-292 (1997)). Total soybean protein content did not change after silencing α and α′ subunits of β-conglycinin by RNAi (Kinney et al., The Plant Cell, 13, 623-629 (2001)). The resulting engineered seeds accumulated more glycinin that accounts for more than 50% of total seed protein, which compensated for the missing β-conglycinin in the engineered seeds.

β-conglycinin consists of 3 isoforms, α, α′ and β. Among them, only α and α′ contain Met and Trp residues in the mature protein. Glycinin has 5 isoforms, all of which have higher Met and Trp content compared to these of β-conglycinin (Utsumi et al., Food Science and Technology, 257-292 (1997)).

Cystathionine-gamma-synthase (CGS) catalyzes the formation of cystathionine that is subsequently converted to homocysteine and finally to methionine (Kreft et al., Plant Physiology, 131, 1843-1854 (2003)). Methionine, the product of CGS, functions not only as a protein storage component, but also as a metabolite in plant cells. In Arabidopsis, CGS expression is regulated at the level of mRNA stability as a feedback mechanism from its product, such as Met or its metabolites. Exon 1 of CGS acts as a Cis regulatory element to down-regulate its own mRNA stability in response to excess accumulation of Met (Chiba et al., Science, 286, 1371-1374 (1999)). There are two CGS genes in soybean, Glyma.09g235400 (GM-CGS1) and Glyma.18g261600 (GM-CGS2)

Lysine is one of the essential amino acids that are present in limiting amounts in crop seeds. The lysine biosynthetic pathway is feedback inhibited by lysine at a rate limiting step, catalyzed by dihydrodipicolinate synthase (DHPS). Seed specific expression of feedback insensitive bacterial DHPS enzyme in various plants resulted in significant seed lysine over production (Falco et al., Bio/Technology, 13, 577-582 (1995); Mazur et al., Science, 285, 372-375 (1999)). The enhanced lysine production may be associated with increased activity of the lysine catabolic enzyme, such as the bi-functional enzyme Lysine-ketoglutarate reductase /Saccharopine dehydrogenase (LKR/SDH) and enhanced levels of lysine catabolic products (Falco et al., Bio/Technology, 13, 577-582 (1995); Mazur et al., Science, 285, 372-375 (1999)). Disclosed are plants, seeds and methods in which blocking the lysine degradation pathway contributes to the accumulation of lysine content in plant cells.

In certain embodiments, the soybean plant or soybean seed comprising the modified glycinin protein comprises a decrease in the expression of beta-conglycinin and a modification increasing the expression of a polynucleotide encoding CGS. In certain embodiments, the soybean plant or soybean seed comprising the modified glycinin protein comprises a decrease in the expression of beta-conglycinin, a modification increasing the expression of a polynucleotide encoding CGS, and a modification decreasing the expression and/or activity of lysine-ketoglutarate reductase /Saccharopine dehydrogenase (LKR/SDH), and/or a modification increasing the activity of dihydrodipicolinate synthase (DHPS).

In certain embodiments, the soybean plant or soybean seed comprising the modified glycinin protein comprises a decrease in the expression of beta-conglycinin and a decrease in the expression and/or activity of MGL. In certain embodiments, the soybean plant or soybean seed comprising the modified glycinin protein comprises a decrease in the expression of beta-conglycinin and a decrease in the expression and/or activity of MGL and a modification decreasing the expression and/or activity of lysine-ketoglutarate reductase /Saccharopine dehydrogenase (LKR/SDH), and/or a modification increasing the activity of dihydrodipicolinate synthase (DHPS).

In certain embodiments, the soybean plant or soybean seed comprising the modified glycinin protein comprises a modification increasing the expression of a polynucleotide encoding CGS and a decrease in the expression and/or activity of MGL. In certain embodiments, the soybean plant or soybean seed comprising the modified glycinin protein comprises a modification increasing the expression of a polynucleotide encoding CGS and a decrease in the expression and/or activity of MGL and a modification decreasing the expression and/or activity of lysine-ketoglutarate reductase /Saccharopine dehydrogenase (LKR/SDH), and/or a modification increasing the activity of dihydrodipicolinate synthase (DHPS).

As used herein, “decrease in expression” “decreased expression” or the like refers to any detectable reduction in expression of a gene and/or the corresponding polypeptide. Similarly, “decrease in activity” “decreased activity” or the like refers to any detectable reduction in the activity (e.g., enzymatic activity) of the encoded polypeptide. The method by which the expression or activity of a gene or polypeptide described herein is decreased is not particularly limited and can be done using methods known in the art such at RNAi, gene knockdown, gene knockout, or targeted amino acid modification.

As used herein a “gene knockout” is used to refer to gene in which there is no detectable expression of the mRNA or protein encoded by the gene, whereas “gene knockdown” is used to refer to a gene in which there is reduced expression of the mRNA or protein encoded by the gene. As used herein, “decreased expression” encompasses both gene knockout and gene knockdown.

As used herein, a “targeted” genetic modification refers to the direct manipulation of an organism’s genes. The targeted modification may be introduced using any technique known in the art, such as, for example, plant breeding, genome editing, or single locus conversion.

In certain embodiments, the modification decreasing the expression of beta-conglycinin comprises a knockout of one or more (e.g., 2 or more, 3 or more, or 4 or more) isoforms of a beta-conglycinin gene. In certain embodiments, the one or more isoforms of the beta-conglycinin gene encodes a beta-conglycinin isoforms comprising an amino acid sequence that is at least 90% identical to any one of SEQ ID NOs: 57-63. In certain embodiments, the modification decreasing the expression of beta-conglycinin comprises a knockout of the beta-conglycinin gene encoding the beta-conglycinin isoforms comprising SEQ ID NOs: 57-63. In certain embodiments, the ratio of glycinin to conglycinin in the soybean plant or soybean seed is at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 65, 70, 75, 80, 85, 90, 95, 100, 500 or 1000 to 1.

In certain embodiments, the modification decreasing the expression and/or activity of MGL comprises a knockout of an MGL gene wherein the MGL gene encodes an MGL protein comprising an amino acid sequence that is at least 90% identical to SEQ ID NO: 53 or 54.

In certain embodiments, the modification decreasing the expression and/or activity of LKR/SDH comprises a knockout of an LKR/SDH gene, wherein the LKR/SDH gene encodes an LKR/SDH protein comprising an amino acid sequence that is at least 90% identical to SEQ ID NO: 55 or 56.

As used herein, “increase in activity” “increased activity” and the like refers to any detectable gain in activity (e.g., enzymatic activity) of the polypeptide. The method by which the activity of a polypeptide described herein is decreased is not particularly limited and can be done using methods known in the art such as increasing expression of the gene encoding the polypeptide (e.g., transgenic expression, promoter swap, gene modification) or a targeted modification the gene encoding the polypeptide to, for example, remove a self-regulatory domain.

In certain embodiments, the method increasing the expression of a polynucleotide encoding CGS comprises a targeted genetic modification that removes a self-regulatory domain of a CGS gene. In certain embodiments, the CGS self-regulatory domain encodes a polypeptide comprising an amino acid sequence that is at least 80% identical to SEQ ID NO: 64. In certain embodiments, the modified CGS gene encodes a CGS protein comprising an amino acid sequence that is at least 70% identical to any one of SEQ ID NOs: 35-37.

In certain embodiments, the soybean plants or soybean seeds further comprise at least one additional modification that increases the total protein in the seed as compared to a control seed (e.g., seed not comprising the at least one modification). In certain embodiments, the introduced modification increases the protein content in the soybean seed at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

In certain embodiments, the soybean plants or soybean seeds further comprise at least one modification decreasing raffinose family oligosaccharides (RFO) content in the seed. In certain embodiments, the modification comprises a decrease in the expression and/or activity of a raffinose synthase. In certain embodiments, the modification comprises a decrease in the expression and/or activity of raffinose synthase 2 (RS2) and/or raffinose synthase 4 (RS4). In certain embodiments, the soybean seed comprises at least a 35%, 40%, 45%, 50%, 55%, 60%, 65%, 70%, 75%, 80%, 85%, 90%, 95%, or 99% decrease in expression of RS2, RS4, or RS2 and RS4, as compared to a control seed. In certain embodiments, the seed comprises less than about 6%, 5.5%, 5%, 4.5%, 4%, 3.5%, 3%, 2.5%, 2%, 1.5%, 1%, or 0.5% RFO content on a dry weight basis. In certain embodiments, the introduced modification decreases RFO content by at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed

Raffinose family oligosaccharides (RFOs) are alpha-galactosyl derivatives of sucrose, and include, for example, raffinose and stachyose. RFOs are anti-nutritional factors that reduce metabolizable energy, cause poor digestibility, and an increase in flatulence and diarrhea in monogastric animals. As used herein, raffinose family oligosaccharides (RFO) content refers to the content of raffinose and stachyose. The RFO content can be measured using methods known in the art such as those described in U.S. Pat. Publication No. 2019-0383733.

### C. Soybean Seeds or Soybean Plants Producing Seeds Comprising an Increase in One or More Essential Amino Acids

The present disclosure further provides soybean seeds or soybean plants producing seeds comprising an increase in the amount of one or more essential amino acids, as compared to a control seed.

In certain embodiments, the soybean seed or soybean plant comprises at least one modification selected from the group consisting of a targeted genetic modification of a glycinin protein to produce a modified glycinin protein comprising an insertion or substitution of one or more methionine, threonine, tryptophan, or lysine residues, or any combination thereof, a modification decreasing the expression of beta-conglycinin, a modification increasing the expression of a polynucleotide encoding a cystathionine-gamma-synthase (CGS), a modification decreasing the expression and/or activity of MGL, a modification decreasing the expression and/or activity of LKR/SDH, and a modification increasing the activity of DHPS, or any combination thereof.

In certain embodiments, the soybean seed or soybean plant comprises at least one modification selected from the group consisting of a targeted genetic modification of a beta-conglycinin protein to produce a modified beta-conglycinin protein comprising an insertion or substitution of one or more methionine, threonine, tryptophan, or lysine residues, or any combination thereof, a targeted genetic modification of a glycinin protein to produce a modified glycinin protein comprising an insertion or substitution of one or more methionine, threonine, tryptophan, or lysine residues, or any combination thereof, a modification increasing the expression of a polynucleotide encoding a cystathionine-gamma-synthase (CGS), a modification decreasing the expression and/or activity of MGL, a modification decreasing the expression and/or activity of LKR/SDH, and a modification increasing the activity of DHPS, or any combination thereof.

In certain embodiments, the soybean seed or the soybean seed of the plant comprises at least about a 10%, 15%, 20%, 25%, 30%, 35%, 40%, 50%, 60%, 70%, 80%, 90%, 100%, or 500% and less than about a 1000%, 500%, 100%, 90%, 80%, 70%, 60%, or 50% increase in the amount of the one or more essential amino acids as compared to a control seed.

In certain embodiments, the one or more essential amino acids is one or more of methionine, cystine, tryptophan, threonine, and lysine, or any combination thereof. In certain embodiments, the one or more essential amino acids is methionine and/or tryptophan. In certain embodiments, the seed produced from the soybean plant or the soybean seed comprises at least a 15% increase in the amount of methionine as compared to a control seed (e.g., an unmodified seed). In certain embodiments, the seed produced from the soybean plant or the soybean seed comprises at least a 15% increase in the amount of tryptophan as compared to a control seed (e.g., an unmodified seed). In certain embodiments, the seed produced from the soybean plant or the soybean seed comprises at least a 15% increase in the amount of methionine and tryptophan as compared to a control seed (e.g., an unmodified seed). In certain embodiments, the seed produced from the soybean plant or the soybean seed comprises at least a 15% increase in the amount of threonine as compared to a control seed (e.g., an unmodified seed). In certain embodiments, the seed produced from the soybean plant or the soybean seed comprises at least a 15% increase in the amount of lysine as compared to a control seed (e.g., an unmodified seed).

In certain embodiments, the soybean seed or the soybean plant comprises a targeted genetic modification of a glycinin protein and a decrease in the expression of beta-conglycinin. In certain embodiments, the soybean seed or the soybean plant comprises a targeted genetic modification of a glycinin protein and a decrease in the expression of beta-conglycinin and a decrease in the expression and/or activity of MGL, a decrease in the expression and/or activity of LKR/SDH, and/or an increase the activity of DHPS.

In certain embodiments, the soybean seed or the soybean plant comprises a decrease in the expression of beta-conglycinin and a modification increasing the expression of a polynucleotide encoding CGS. In certain embodiments, the soybean seed or the soybean plant comprises a decrease in the expression of beta-conglycinin and a modification increasing the expression of a polynucleotide encoding CGS and a decrease in the expression and/or activity of MGL, a decrease in the expression and/or activity of LKR/SDH, and/or an increase the activity of DHPS.

In certain embodiments, the soybean seed or the soybean plant comprises a targeted genetic modification of a glycinin protein and a modification increasing the expression of a polynucleotide encoding CGS. In certain embodiments, the soybean seed or the soybean plant comprises a targeted genetic modification of a glycinin protein and a modification increasing the expression of a polynucleotide encoding CGS and a decrease in the expression and/or activity of MGL, a decrease in the expression and/or activity of LKR/SDH, and/or an increase the activity of DHPS.

In certain embodiments, the soybean seed or the soybean plant comprises a targeted genetic modification of a glycinin protein, a decrease in the expression of beta-conglycinin, and a modification increasing the expression of a polynucleotide encoding CGS. In certain embodiments, the soybean seed or the soybean plant comprises a targeted genetic modification of a glycinin protein, a decrease in the expression of beta-conglycinin, and a modification increasing the expression of a polynucleotide encoding CGS and a decrease in the expression and/or activity of MGL, a decrease in the expression and/or activity of LKR/SDH, and/or an increase the activity of DHPS.

In certain embodiments, the soybean seed or the soybean plant comprises a decrease in the expression and/or activity of LKR/SDH and an increase the activity of DHPS. In certain embodiments, the soybean seed or the soybean plant comprises a decrease in the expression and/or activity of LKR/SDH and an increase the activity of DHPS and a decrease in the expression and/or activity of MGL.

In certain embodiments, the soybean seed or the soybean plant comprises a targeted genetic modification of a glycinin protein and one or more of a modification decreasing the expression of beta-conglycinin, a modification increasing the expression of a polynucleotide encoding a cystathionine-gamma-synthase (CGS), a modification decreasing the expression and/or activity of MGL, a modification decreasing the expression and/or activity of LKR/SDH, or a modification increasing the activity of DHPS.

In certain embodiments, the soybean seed or the soybean plant comprises a modification decreasing the expression of beta-conglycinin and one or more of a targeted genetic modification of a glycinin protein, a modification increasing the expression of a polynucleotide encoding CGS, a modification decreasing the expression and/or activity of MGL, a modification decreasing the expression and/or activity of LKR/SDH, or a modification increasing the activity of DHPS.

In certain embodiments, the soybean seed or the soybean plant comprises a modification increasing the expression of a polynucleotide encoding CGS and one or more of a targeted genetic modification of a glycinin protein, a modification decreasing the expression of beta-conglycinin, a modification decreasing the expression and/or activity of MGL, a modification decreasing the expression and/or activity of LKR/SDH, or a modification increasing the activity of DHPS.

In certain embodiments, the soybean seed or the soybean plant comprises a modification decreasing the expression and/or activity of MGL and one or more of a targeted genetic modification of a glycinin protein, a modification decreasing the expression of beta-conglycinin, a modification increasing the expression of a polynucleotide encoding a cystathionine-gamma-synthase (CGS), a modification decreasing the expression and/or activity of LKR/SDH, or a modification increasing the activity of DHPS.

In certain embodiments, the soybean seed or the soybean plant comprises a modification decreasing the expression and/or activity of LKR/SDH and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification increasing the activity of DHPS.

In certain embodiments, the soybean seed or the soybean plant comprises a modification increasing the activity of DHPS and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification decreasing the expression and/or activity of LKR/SDH.

In certain embodiments, the modified glycinin protein comprises an amino acid sequence that is at least 80% identical to SEQ ID NO: 1.

In certain embodiments, the modified glycinin protein comprising an amino acid sequence that is at least 80% identical to SEQ ID NO: 1 comprises at least a 40% increase in the amount of methionine as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of tryptophan as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of both methionine and tryptophan as a percent of the total amino acid of the glycinin protein of SEQ ID NO: 1.

In certain embodiments, the modified glycinin protein comprising an amino acid sequence that is at least 80% identical to SEQ ID NO: 1 glycinin protein comprises at least a 40% increase in the amount of threonine as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of lysine as a percent of the total amino acid of the glycinin protein of SEQ ID NO: 1.

In certain embodiments, the modified glycinin protein comprising an amino acid sequence that is at least 80% identical to SEQ ID NO: 1 comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, and 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof.

In certain embodiments, the modified glycinin protein comprising an amino acid sequence that is at least 80% identical to SEQ ID NO: 1 comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, or any combination thereof.

In certain embodiments, the modified glycinin protein comprising an amino acid sequence that is at least 80% identical to SEQ ID NO: 1 comprises P300WWWWWWW.

In certain embodiments, the modified glycinin protein comprising an amino acid sequence that is at least 80% identical to SEQ ID NO: 1 Q196M, Q197M, E198M, Q199M, and Q203M.

In certain embodiments, the modified glycinin protein comprising an amino acid sequence that is at least 80% identical to SEQ ID NO: 1 comprises 486MMMMMM487.

In certain embodiments, the modified glycinin protein comprising an amino acid sequence that is at least 80% identical to SEQ ID NO: 1 comprises Q118MMM, Q119MMM, Q196M, Q197M, E198M, Q199M, Q203M, Q272M, Q274M, 276M, E278M, E280M, E281M, E282M, E283M, E284M, D285M, E286M, P288M, Q289M, P300MMMMMMM, and 486MMMMMM487.

In certain embodiments, the modified glycinin protein comprising an amino acid sequence that is at least 80% identical to SEQ ID NO: 1 comprises L39M, L51M, L74M, L141M, L175M, L193M, L226M, L262M, L321M, L347M, L376M, L385M, and L390M.

In certain embodiments, the modified glycinin protein comprising an amino acid sequence that is at least 80% identical to SEQ ID NO: 1 comprises L39M, L51M, L74M, L141M, L175M, L193M, L226M, L262M, L321M, L347M, L376M, L385M, L390M, Y104W, Y134W, Y153W, Y334W, F361W, Y431W, and F480W.

In certain embodiments, the modified glycinin protein comprising an amino acid sequence that is at least 80% identical to SEQ ID NO: 1 comprises Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, 486MMMMMM487, Y104W, Y134W, Y153W, Y334W, F361W, Y431W, and F480W.

The modification decreasing the expression of beta-conglycinin may be any modification of beta-conglycinin described herein, such as, for example, a knockout of one or more isoforms of beta-conglycinin. In certain embodiments, the ratio of glycinin to conglycinin in the soybean plant or soybean seed is at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 65, 70, 75, 80, 85, 90, 95, 100, 500 or 1000 to 1.

The modification increasing the expression of a polynucleotide encoding CGS may be any modification of the CGS gene described herein, such as, for example, a targeted genetic modification that removes a self-regulatory domain of the CGS gene.

The modification decreasing the expression of MGL may be any modification of MGL described herein, such as, for example, a knockout of the MGL gene.

The modification decreasing the expression of LKR/SDH may be any modification of LKR/SDH described herein, such as, for example, a knockout of the LKR/SDH gene.

In certain embodiments, the modification increasing the activity of DHPS comprises a targeted genetic modification of the DHPS gene to remove a feedback inhibition domain of a DHPS gene.

In certain embodiments, the soybean plants or soybean seeds further comprise at least one additional modification that increases the total protein in the seed as compared to a control seed (e.g., seed not comprising the at least one modification). In certain embodiments, the soybean seed comprising the at least one modification comprises at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

In certain embodiments, the soybean plants or soybean seeds further comprise at least one additional modification decreasing the raffinose family oligosaccharides (RFO) content in the seed. In certain embodiments, the modification comprises a decrease in the expression and/or activity of a raffinose synthase. In certain embodiments, the modification comprises a decrease in the expression and/or activity of raffinose synthase 2 (RS2) and/or raffinose synthase 4 (RS4). In certain embodiments, the soybean seed comprises at least a 35%, 40%, 45%, 50%, 55%, 60%, 65%, 70%, 75%, 80%, 85%, 90%, 95%, or 99% decrease in expression of RS2, RS4, or RS2 and RS4, as compared to a control seed. In certain embodiments, the seed comprises less than about 6%, 5.5%, 5%, 4.5%, 4%, 3.5%, 3%, 2.5%, 2%, 1.5%, 1%, or 0.5% RFO content on a dry weight basis. In certain embodiments, the introduced modification decreases RFO content by at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

### D. Soybean Seeds or Soybean Plants Producing Seeds Comprising an Increase in Methionine

The present disclosure further provides soybean seeds or soybean plants producing seeds comprising at least about a 10%, 12%, 14%, 16%, 18%, 20%, 22%, 24%, 26%, 28%, 30% and less than about a 40%, 38%, 36%, 34%, 32%, 30%, 28%, 26%, 24%, 22% or 20% increase in methionine on a dry weight of seed basis, as compared to a control seed. In certain embodiments, the soybean plant or soybean seed further comprises at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in protein measured on a dry weight basis, as compared to a control seed.

As used herein, “percentage point” (pp) difference, change, increase or decrease refers to the arithmetic difference of two percentages, e.g. [transgenic or genetically modified value (%) -control value (%)] = percentage points. For example, a modified seed may contain 20% by weight of a component and the corresponding unmodified control seed may contain 15% by weight of that component. The difference in the component between the control and transgenic seed would be expressed as 5 percentage points.

In certain embodiments, the soybean seed comprises at least one modification selected from the group consisting of a targeted genetic modification of a glycinin protein to produce a modified glycinin protein comprising an insertion or substitution of one or more methionine, threonine, tryptophan, or lysine residues, or any combination thereof, a modification decreasing the expression of beta-conglycinin, a modification increasing the expression of a polynucleotide encoding cystathionine-gamma-synthase (CGS), a modification decreasing the expression and/or activity of MGL, a modification decreasing the expression and/or activity of LKR/SDH, and a modification increasing the activity of DHPS, or any combination thereof.

The combination of modifications may be any modification described herein.

In certain embodiments, the soybean seed or the soybean plant comprises a targeted genetic modification of a glycinin protein and one or more of a modification decreasing the expression of beta-conglycinin, a modification increasing the expression of a polynucleotide encoding CGS, a modification decreasing the expression and/or activity of MGL, a modification decreasing the expression and/or activity of LKR/SDH, or a modification increasing the activity of DHPS.

In certain embodiments, the soybean seed or the soybean plant comprises a modification decreasing the expression of beta-conglycinin and one or more of a targeted genetic modification of a glycinin protein, a modification increasing the expression of a polynucleotide encoding CGS, a modification decreasing the expression and/or activity of MGL, a modification decreasing the expression and/or activity of LKR/SDH, or a modification increasing the activity of DHPS.

In certain embodiments, the soybean seed or the soybean plant comprises a modification increasing the expression of a polynucleotide encoding CGS and one or more of a targeted genetic modification of a glycinin protein, a modification decreasing the expression of beta-conglycinin, a modification decreasing the expression and/or activity of MGL, a modification decreasing the expression and/or activity of LKR/SDH, or a modification increasing the activity of DHPS.

In certain embodiments, the soybean seed or the soybean plant comprises a modification decreasing the expression and/or activity of MGL and one or more of a targeted genetic modification of a glycinin protein, a modification decreasing the expression of beta-conglycinin, a modification increasing the expression of a polynucleotide encoding CGS, a modification decreasing the expression and/or activity of LKR/SDH, or a modification increasing the activity of DHPS.

In certain embodiments, the soybean seed or the soybean plant comprises a modification decreasing the expression and/or activity of LKR/SDH and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification increasing the activity of DHPS.

In certain embodiments, the soybean seed or the soybean plant comprises a modification increasing the activity of DHPS and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification decreasing the expression and/or activity of LKR/SDH.

In certain embodiments, the modified glycinin protein comprises an amino acid sequence that is at least 80% identical to SEQ ID NO: 1.

In certain embodiments, the modified glycinin protein comprising an amino acid sequence that is at least 80% identical to SEQ ID NO: 1 comprises at least a 40% increase in the amount of methionine as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of tryptophan as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of both methionine and tryptophan as a percent of the total amino acid of the glycinin protein of SEQ ID NO: 1.

In certain embodiments, the modified glycinin protein comprising an amino acid sequence that is at least 80% identical to SEQ ID NO: 1 glycinin protein comprises at least a 40% increase in the amount of threonine as a percent of the total amino acid of the glycinin protein of SEQ ID NO: 1.

In certain embodiments, the modified glycinin protein comprising an amino acid sequence that is at least 80% identical to SEQ ID NO: 1 comprises at least a 40% increase in the amount of lysine as a percent of the total amino acid of the glycinin protein of SEQ ID NO: 1.

In certain embodiments, the modified glycinin protein comprising an amino acid sequence that is at least 80% identical to SEQ ID NO: 1 comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, and 486MMMMMM487, an insertion of one or more methionine residues, one or more tryptophan residues, one or more threonine residues, one or more lysine residues, or any combination thereof between amino acids F20 and Q28 of the glycinin protein of SEQ ID NO: 1, an insertion of one or more methionine residues, one or more tryptophan residues, one or more threonine residues, one or more lysine residues, or any combination thereof between amino acids F111 and R129 of the glycinin protein of SEQ ID NO: 1, an insertion of one or more methionine residues, one or more tryptophan residues, one or more threonine residues, one or more lysine residues, or any combination thereof between amino acids Y195 and E214 of the glycinin protein of SEQ ID NO: 1, an insertion of one or more methionine residues, one or more tryptophan residues, one or more threonine residues, one or more lysine residues, or any combination thereof between amino acids K266 and N310 of the glycinin protein of SEQ ID NO: 1, an insertion of one or more methionine residues, one or more tryptophan residues, one or more threonine residues, one or more lysine residues, or any combination thereof between amino acids E468 and A495 of the glycinin protein of SEQ ID NO: 1, or any combination thereof.

In certain embodiments, the modified glycinin protein comprising an amino acid sequence that is at least 80% identical to SEQ ID NO: 1 comprises at least one amino acid modification selected from the group consisting of Q196M, Q197M, E198M, Q199M, Q203M, Q272M, Q274M, P276M, E278M, E280M, E281M, E282M, E283M, E284M, D285M, E286M, P288M, Q289M, L39M, L51M, L74M, L141M, L175M, L193M, L226M, L262M, L321M, L347M, L376M, L385M, L390M, Y104W, Y134W, Y153W, Y334W, F361W, Y431W, F480W, Q118MMM, Q119MMM, P300WWWWWWW, P300MMMMMMM, 486MMMMMM487, or any combination thereof.

In certain embodiments, the modified glycinin protein comprising an amino acid sequence that is at least 80% identical to SEQ ID NO: 1 comprises P300WWWWWWW.

In certain embodiments, the modified glycinin protein comprising an amino acid sequence that is at least 80% identical to SEQ ID NO: 1 Q196M, Q197M, E198M, Q199M, and Q203M.

In certain embodiments, the modified glycinin protein comprising an amino acid sequence that is at least 80% identical to SEQ ID NO: 1 comprises 486MMMMMM487.

In certain embodiments, the modified glycinin protein comprising an amino acid sequence that is at least 80% identical to SEQ ID NO: 1 comprises Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, and 486MMMMMM487.

In certain embodiments, the modified glycinin protein comprising an amino acid sequence that is at least 80% identical to SEQ ID NO: 1 comprises L39M, L51M, L74M, L141M, L175M, L193M, L226M, L262M, L321M, L347M, L376M, L385M, and L390M.

In certain embodiments, the modified glycinin protein comprising an amino acid sequence that is at least 80% identical to SEQ ID NO: 1 comprises L39M, L51M, L74M, L141M, L175M, L193M, L226M, L262M, L321M, L347M, L376M, L385M, L390M, Y104W, Y134W, Y153W, Y334W, F361W, Y431W, and F480W.

In certain embodiments, the modified glycinin protein comprising an amino acid sequence that is at least 80% identical to SEQ ID NO: 1 comprises Q118MMM, Q119MMM, Q196M, Q197M, E198M, Q199M, Q203M, Q272M, Q274M, P276M, E278M, E280M, E281M, E282M, E283M, E284M, D285M, E286M, P288M, Q289M, P300MMMMMMM, 486MMMMMM487, Y104W, Y134W, Y153W, Y334W, F361W, Y431W, and F480W.

The modification decreasing the expression of beta-conglycinin may be any modification of beta-conglycinin described herein, such as, for example, a knockout of one or more isoforms of beta-conglycinin. In certain embodiments, the ratio of glycinin to conglycinin in the soybean plant or soybean seed is at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 65, 70, 75, 80, 85, 90, 95, 100, 500 or 1000 to 1.

The modification increasing the expression of a polynucleotide encoding CGS may be any modification of the CGS gene described herein, such as, for example, a targeted genetic modification that removes a self-regulatory domain of the CGS gene.

The modification decreasing the expression of MGL may be any modification of MGL described herein, such as, for example, a knockout of the MGL gene.

The modification decreasing the expression of LKR/SDH may be any modification of LKR/SDH described herein, such as, for example, a knockout of the LKR/SDH gene. In certain embodiments, the modification increasing the activity of DHPS comprises a targeted genetic modification of the DHPS gene to remove a feedback inhibition domain of a DHPS gene.

In certain embodiments, the soybean plants or soybean seeds further comprise one or more modifications that produce an altered seed composition. For example, the soybean plants or seeds may comprise a modification that increases the total protein in the seed as compared to a control seed (e.g., seed not comprising the at least one modification). The soybean plants or soybean seeds may comprise at least one modification decreasing the raffinose family oligosaccharides (RFO) content in the seed, such as by decreasing the expression and/or activity of a raffinose synthase. Soybean plants and seeds comprising modifications altering the amino acid profile of seed storage proteins may further comprise one or both of the modifications increasing total protein and decreasing the RFO content in the seed. As used herein, raffinose family oligosaccharides (RFO) refers to the alpha-galactosyl derivatives of sucrose, and include, for example raffinose and stachyose. The RFO content can be measured using methods known in the art such as those described in U.S. Pat. Publication No. 2019-0383733.

In certain embodiments, the soybean seed comprising the at least one modification increasing total seed protein comprises at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

In certain embodiments, the modification comprises a decrease in the expression and/or activity of raffinose synthase 2 (RS2) and/or raffinose synthase 4 (RS4). In certain embodiments, the soybean seed comprises at least a 35%, 40%, 45%, 50%, 55%, 60%, 65%, 70%, 75%, 80%, 85%, 90%, 95%, or 99% decrease in expression of RS2, RS4, or RS2 and RS4, as compared to a control seed. In certain embodiments, the seed comprises less than about 6%, 5.5%, 5%, 4.5%, 4%, 3.5%, 3%, 2.5%, 2%, 1.5%, 1%, or 0.5% RFO content on a dry weight basis. In certain embodiments, the introduced modification decreases RFO content by at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

### E. Soy Protein Composition

The disclosure also provides soy protein compositions having an essential amino acid content of at least 35%, 40%, 45%, 50%, 55%, 60%, 65%, 70%, 75%, 80%, or 85% and less than 95%, 90%, 85%, 80%, 75%, 70%, 65%, 60%, 55%, 50%, or 45%. In certain embodiments, the soy protein composition comprises at least one, at least 2, at least 3, at least 4, at least 5 characteristic(s) selected from the group consisting of (a) a modified glycinin protein comprising at least a 5%, 10%, 15%, 20%, 25%, 30% or 50% increase in the proportion of methionine residues, tryptophan residues or a combination thereof of the total glycinin amino acid residues as compared with a control glycinin protein; (b) comprises less than 15%, 14%, 13%, 12%, 11%, 10%, 9%, 8%, 7%, 6%, 5%, 4%, 3%, 2%, 1%, or 0.5% beta-conglycinin; (c) a CGS protein comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%identical to any one of SEQ ID NOs: 35-37; (d) the composition lacks MGL; (e) the composition lacks LKR/SDH; (f) a modified DHPS protein; and (g) comprises less than about 6%, 5.5%, 5%, 4.5%, 4%, 3.5%, 3%, 2.5%, 2%, 1.5%, 1%, or 0.5% RFO.

The modified glycinin protein of the soy protein composition may be any modified glycinin protein described herein.

The soy protein composition may lack any of the beta-conglycinin isoforms described herein (e.g., SEQ ID NOs: 57-63). In certain embodiments, the soy protein composition lacks more than one (e.g., 2, 3, 4, or 5) beta-conglycinin isoform.

The modified CGS protein of the soy protein composition may be any modified CGS protein described herein. In certain embodiments, the modified CGS protein comprises an amino acid sequence that is at least 90% identical to the amino acid sequence of any one of SEQ ID NOs: 35-37

The soy protein may lack any MGL gene described herein. Similarly, the soy protein composition may lack any LKR/SDH gene described herein.

The modified DHPS protein of the soy protein composition may be any modified DHPS protein described herein.

In certain embodiments, the sum of the methionine and tryptophan in the soy protein composition is greater than about 20, 25, 30, 35, 40, 45, 50, 55, 60, 65, 70, 75, 80, 85, 90, 95, 100, 125, 150, 175, or 200 mg/g protein and less than 500, 400, 300, 250, 200, 150, 100, 75, 50, or 40 mg/g protein. In certain embodiments, the sum of the methionine, lysine, threonine and tryptophan in the soy protein composition is greater than about 20, 25, 30, 35, 40, 45, 50, 55, 60, 65, 70, 75, 80, 85, 90, 95, 100, 125, 150, 175, or 200 mg/g protein and less than 500, 400, 300, 250, 200, 150, 100, 75, 50, or 40 mg/g protein.

As used herein, “soy protein composition” refers to food ingredients for humans or animals which contain soy proteins. In certain embodiments, the composition is an animal feed composition. In certain embodiments, the composition is a human food composition. In certain embodiments, the human food composition is a composition selected from the group consisting of soybean meal; soyflour; defatted soyflour; soymilk; spray-dried soymilk; soy protein concentrate; texturized soy protein concentrate; hydrolyzed soy protein; soy protein isolate; spray-dried tofu; soy meat analog; soy cheese analog; and soy coffee creamer.

Synthetic alternatives to essential amino acids may be used as supplements in animal feed to drive animal productivity. Provided are methods and feeds for feeding animals in which the feed contains a soy protein composition described herein which contains increased concentrations or amounts of one or more essential amino acids. Such protein compositions may be fed to animals, such as pigs or chickens, in a feeding regimen which does not require a synthetic or manufactured amino acid supplement to maintain animal growth compared with a control soy protein composition from comparable unmodified soybeans.

### II. Methods

### A. Method for Generating Soybean Plants Producing Seeds With an Increased Essential Amino Acid Content

The present disclosure further provides a method of generating soybean plants producing seeds with an increased essential amino acid content comprising introducing into a regenerable plant cell a recombinant DNA construct described herein, at least one targeted genetic modification selected from the group consisting of a targeted genetic modification of a glycinin protein to produce a modified glycinin protein comprising an insertion or substitution of one or more methionine, threonine, tryptophan, or lysine residues, or any combination thereof, a modification decreasing the expression of beta-conglycinin, a modification increasing the expression of a polynucleotide encoding a cystathionine-gamma-synthase (CGS), a modification decreasing the expression and/or activity of MGL, a modification decreasing the expression and/or activity of LKR/SDH, or a modification increasing the activity of DHPS, or any combination thereof; and generating the plant, wherein the plant comprises the at least one modification. The modified glycinin protein of the methods may be any modified glycinin protein described herein.

In certain embodiments, the method generates plants producing seed comprising at least about a 10%, 15%, 20%, 25%, 30%, 35%, 40%, 50%, 60%, 70%, 80%, 90%, 100%, or 500% and less than about a 1000%, 500%, 100%, 90%, 80%, 70%, 60%, or 50% increase in the amount of one or more essential amino acids as compared to a control seed.

As used herein, “percent increase” refers to a change or difference expressed as a fraction of the control value, e.g. {[modified/transgenic/test value (%) - control value (%)]/control value (%)} x 100% = percent change., or {[value obtained in a first location (%) - value obtained in second location (%)]/ value in the second location (%)}x100 = percent change.

In certain embodiments, the one or more essential amino acids is one or more of methionine, cystine, tryptophan, threonine, and lysine, or any combination thereof. In certain embodiments, the one or more essential amino acids is methionine and/or tryptophan.

In certain embodiments, the method generates plants producing seed comprising at least a 15%, 20%, 25%, 30%, 35%, 40%, 50%, 60%, 70%, 80%, 90%, or 100% and less than about a 150%, 100% 90%, 80%, 70%, 60%, 50%, 40%, 35%, 30% or 25% increase in the amount of methionine as compared to a control seed (e.g., an unmodified seed).

In certain embodiments, the method generates plants producing seed comprising at least a 15%, 20%, 25%, 30%, 35%, 40%, 50%, 60%, 70%, 80%, 90%, or 100% and less than about a 150%, 100% 90%, 80%, 70%, 60%, 50%, 40%, 35%, 30% or 25% increase in the amount of tryptophan as compared to a control seed (e.g., an unmodified seed).

In certain embodiments, the method generates plants producing seed comprising at least a 15%, 20%, 25%, 30%, 35%, 40%, 50%, 60%, 70%, 80%, 90%, or 100% and less than about a 150%, 100% 90%, 80%, 70%, 60%, 50%, 40%, 35%, 30% or 25% increase in the amount of methionine and tryptophan as compared to a control seed (e.g., an unmodified seed).

In certain embodiments, the method generates plants producing seed comprising at least a 15%, 20%, 25%, 30%, 35%, 40%, 50%, 60%, 70%, 80%, 90%, or 100% and less than about a 150%, 100% 90%, 80%, 70%, 60%, 50%, 40%, 35%, 30% or 25% increase in the amount of threonine as compared to a control seed (e.g., an unmodified seed).

In certain embodiments, the method generates plants producing seed comprising at least a 15%, 20%, 25%, 30%, 35%, 40%, 50%, 60%, 70%, 80%, 90%, or 100% and less than about a 150%, 100% 90%, 80%, 70%, 60%, 50%, 40%, 35%, 30% or 25% increase in the amount of lysine as compared to a control seed (e.g., an unmodified seed).

The modification decreasing the expression of beta-conglycinin may be any modification of beta-conglycinin described herein. The modification of a CGS gene may be any modification of a CGS gene described herein. The modification decreasing the expression and/or activity of MGL may be any modification of MGL described herein. The modification decreasing the expression and/or activity of LKR/SDH may be any modification of LKR/SDH described herein. The modification increasing the activity of DHPS may be any modification of DHPS described herein.

In certain embodiments, the method comprises introducing a targeted genetic modification of a glycinin protein and a modification decreasing the expression of beta-conglycinin. In certain embodiments, the method comprises introducing a targeted genetic modification of a glycinin protein and a modification decreasing the expression of beta-conglycinin and a modification decreasing the expression and/or activity of MGL, a modification decreasing the expression and/or activity of LKR/SDH and/or a modification increasing the activity of DHPS.

In certain embodiments, the method comprises introducing a targeted genetic modification of a glycinin protein and a modification increasing the expression of a polynucleotide encoding CGS. In certain embodiments, the method comprises introducing a targeted genetic modification of a glycinin protein and a modification increasing the expression of a polynucleotide encoding CGS and a modification decreasing the expression and/or activity of MGL, a modification decreasing the expression and/or activity of LKR/SDH and/or a modification increasing the activity of DHPS.

In certain embodiments, the method comprises introducing a modification decreasing the expression of beta-conglycinin and a modification increasing the expression of a polynucleotide encoding CGS. In certain embodiments, the method comprises introducing a modification decreasing the expression of beta-conglycinin and a modification increasing the expression of a polynucleotide encoding CGS and a modification decreasing the expression and/or activity of MGL, a modification decreasing the expression and/or activity of LKR/SDH and/or a modification increasing the activity of DHPS.

In certain embodiments, the method comprises introducing a targeted genetic modification of a glycinin protein, a modification decreasing the expression of beta-conglycinin, and a modification increasing the expression of a polynucleotide encoding CGS. In certain embodiments, the method comprises introducing a targeted genetic modification of a glycinin protein, a modification decreasing the expression of beta-conglycinin, and a modification increasing the expression of a polynucleotide encoding CGS and a modification decreasing the expression and/or activity of MGL, a modification decreasing the expression and/or activity of LKR/SDH and/or a modification increasing the activity of DHPS.

In certain embodiments, the method comprises introducing a modification decreasing the expression and/or activity of LKR/SDH and a modification increasing the activity of DHPS. In certain embodiments, the method comprises introducing a modification decreasing the expression and/or activity of LKR/SDH and a modification increasing the activity of DHPS and a modification decreasing the expression and/or activity of MGL.

In certain embodiments, the method comprises introducing a targeted genetic modification of a glycinin protein and one or more of a modification decreasing the expression of beta-conglycinin, a modification increasing the expression of a polynucleotide encoding CGS, a modification decreasing the expression and/or activity of MGL, a modification decreasing the expression and/or activity of LKR/SDH, or a modification increasing the activity of DHPS.

In certain embodiments, the method comprises introducing a modification decreasing the expression of beta-conglycinin and one or more of a targeted genetic modification of a glycinin protein, a modification increasing the expression of a polynucleotide encoding CGS, a modification decreasing the expression and/or activity of MGL, a modification decreasing the expression and/or activity of LKR/SDH, or a modification increasing the activity of DHPS.

In certain embodiments, the method comprises introducing a modification increasing the expression of a polynucleotide encoding CGS and one or more of a targeted genetic modification of a glycinin protein, a modification decreasing the expression of beta-conglycinin, a modification decreasing the expression and/or activity of MGL, a modification decreasing the expression and/or activity of LKR/SDH, or a modification increasing the activity of DHPS.

In certain embodiments, the method comprises introducing a modification decreasing the expression and/or activity of MGL and one or more of a targeted genetic modification of a glycinin protein, a modification decreasing the expression of beta-conglycinin, a modification increasing the expression of a polynucleotide encoding CGS, a modification decreasing the expression and/or activity of LKR/SDH, or a modification increasing the activity of DHPS.

In certain embodiments, the method comprises introducing a modification decreasing the expression and/or activity of LKR/SDH and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification increasing the activity of DHPS.

In certain embodiments, the method comprises introducing a modification increasing the activity of DHPS and one or more of a targeted genetic modification of a glycinin protein, a modification decreasing the expression of beta-conglycinin, a modification increasing the expression of a polynucleotide encoding CGS, a modification decreasing the expression and/or activity of MGL, or a modification decreasing the expression and/or activity of LKR/SDH.

In certain embodiments, the method further comprises introducing at least one additional modification increasing the total protein in the seed as compared to a control seed (e.g., seed not comprising the at least one modification). In certain embodiments, the introduced modification increases the protein content in the soybean seed at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed.

In certain embodiments, the method further comprises introducing at least one modification decreasing the raffinose family oligosaccharides (RFO) content in the seed. In certain embodiments, the modification comprises a decrease in the expression and/or activity of a raffinose synthase. In certain embodiments, the modification comprises a decrease in the expression and/or activity of raffinose synthase 2 (RS2) and/or raffinose synthase 4 (RS4). In certain embodiments, the soybean seed comprises at least a 35%, 40%, 45%, 50%, 55%, 60%, 65%, 70%, 75%, 80%, 85%, 90%, 95%, or 99% decrease in expression of RS2, RS4, or RS2 and RS4, as compared to a control seed. In certain embodiments, the seed comprises less than about 6%, 5.5%, 5%, 4.5%, 4%, 3.5%, 3%, 2.5%, 2%, 1.5%, 1%, or 0.5% RFO content on a dry weight basis. In certain embodiments, the introduced modification decreases RFO content by at least about a 1%, 1.5%, 2%, 2.5%, 3%, 3.5%, 4%, 5%, 10%, or 15% and less than 20%, 15%, 10%, 9%, 8%, 7%, 6%, or 5% percentage point increase in total protein measured on a dry weight basis, as compared to a control seed

In certain embodiments, the method comprises: (a) providing a guide RNA, at least one polynucleotide modification template, and at least one Cas endonuclease to a plant cell, wherein the at least one Cas endonuclease introduces a double stranded break at an endogenous gene to be modified (e.g., glycinin, beta-conglycinin, CGS, etc) in the plant cell, and wherein the polynucleotide modification template generates a modified gene that encodes any of the polypeptides described herein; (b) obtaining a plant from the plant cell; and (c) generating a progeny plant.

In certain embodiments, the modification is of the endogenous gene (e.g., glycinin, beta-conglycinin, CGS, MGL, LKR/SDH and DHPS). As used herein “endogenous gene” refers to a gene that is original to a host plant and can be used synonymously with “host genomic DNA,” “pre-existing DNA,” and the like. Moreover, for the purposes herein, an endogenous gene includes coding DNA and genomic DNA within and surrounding the coding DNA, such as for example, the promoter, intron, and terminator sequences.

Methods to modify or alter endogenous genomic DNA are known in the art. For example, a pre-existing or endogenous sequence in a host plant can be modified or altered in a site-specific fashion using one or more site-specific engineering systems.

Methods and compositions are provided herein for modifying naturally occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, DC)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks and includes homologous recombination (HR) and single-strand annealing (SSA) (Lieber. 2010 Annu. Rev. Biochem. 79:181-211).

In addition to the double-strand break inducing agents, site-specific base conversions can also be achieved to engineer one or more nucleotide changes to create one or more modifications described herein into the genome. These include for example, a site-specific base edit mediated by an C•G to T•A or an A•T to G•C base editing deaminase enzymes (Gaudelli et al., Programmable base editing of A•T to G•C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4.

In the methods described herein, the endogenous gene may be modified by a CRISPR associated (Cas) endonuclease, a Zn-finger nuclease-mediated system, a meganuclease-mediated system, an oligonucleobase-mediated system, or any gene modification system known to one of ordinary skill in the art.

In certain embodiments the endogenous gene is modified by a CRISPR associated (Cas) endonuclease.

Class I Cas endonucleases comprise multisubunit effector complexes (Types I, III, and IV), while Class 2 systems comprise single protein effectors (Types II, V, and VI) (Makarova et al. 2015, Nature Reviews Microbiology Vol. 13:1-15; Zetsche et al., 2015, Cell 163, 1-13; Shmakov et al., 2015, Molecular Cell 60, 1-13; Haft et al., 2005, Computational Biology, PLoS Comput Biol 1(6): e60; and Koonin et al. 2017, Curr Opinion Microbiology 37:67-78). In Class 2 Type II systems, the Cas endonuclease acts in complex with a guide polynucleotide.

Accordingly, in certain embodiments of the methods described herein the Cas endonuclease forms a complex with a guide polynucleotide (e.g., guide polynucleotide/Cas endonuclease complex).

As used herein, the term “guide polynucleotide”, relates to a polynucleotide sequence that can form a complex with a Cas endonuclease, including the Cas endonucleases described herein, and enables the Cas endonuclease to recognize, optionally bind to, and optionally cleave a DNA target site. The guide polynucleotide sequence can be an RNA sequence, a DNA sequence, or a combination thereof (a RNA-DNA combination sequence). The guide polynucleotide may further comprise a chemically modified base, such as, but not limited, to Locked Nucleic Acid (LNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization.

In certain embodiments, the Cas endonuclease forms a complex with a guide polynucleotide (e.g., gRNA) that directs the Cas endonuclease to cleave the DNA target to enable target recognition, binding, and cleavage by the Cas endonuclease. The guide polynucleotide (e.g., gRNA) may comprise a Cas endonuclease recognition (CER) domain that interacts with the Cas endonuclease, and a Variable Targeting (VT) domain that hybridizes to a nucleotide sequence in a target DNA. In certain embodiments, the guide polynucleotide (e.g., gRNA) comprises a CRISPR nucleotide (crNucleotide; e.g., crRNA) and a trans-activating CRISPR nucleotide (tracrNucleotide; e.g., tracrRNA) to guide the Cas endonuclease to its DNA target. The guide polynucleotide (e.g., gRNA) comprises a spacer region complementary to one strand of the double strand DNA target and a region that base pairs with the tracrNucleotide (e.g., tracrRNA), forming a nucleotide duplex (e.g. RNA duplex).

In certain embodiments, the gRNA is a “single guide RNA” (sgRNA) that comprises a synthetic fusion of crRNA and tracrRNA. In many systems, the Cas endonuclease-guide polynucleotide complex recognizes a short nucleotide sequence adjacent to the target sequence (protospacer), called a “protospacer adjacent motif” (PAM).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In one embodiment, the nucleotide sequence linking the crNucleotide and the tracrNucleotide of a single guide polynucleotide can comprise a tetraloop sequence, such as, but not limiting to a GAAA tetraloop sequence.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

A “protospacer adjacent motif” (PAM) as used herein refers to a short nucleotide sequence adjacent to a target sequence (protospacer) that is recognized (targeted) by a guide polynucleotide/Cas endonuclease system described herein. In certain embodiments, the Cas endonuclease may not successfully recognize a target DNA sequence if the target DNA sequence is not adjacent to, or near, a PAM sequence. In certain embodiments, the PAM precedes the target sequence (e.g. Cas12a). In certain embodiments, the PAM follows the target sequence (e.g. S. pyogenes Cas9). The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “polynucleotide-guided endonuclease”, and “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13). In certain embodiments, the guide polynucleotide/Cas endonuclease complex is provided as a ribonucleoprotein (RNP), wherein the Cas endonuclease component is provided as a protein and the guide polynucleotide component is provided as a ribonucleotide.

Examples of Cas endonucleases for use in the methods described herein include, but are not limited to, Cas9 and Cpf1. Cas9 (formerly referred to as Cas5, Csn1, or Csx12) is a Class 2 Type II Cas endonuclease (Makarova et al. 2015, Nature Reviews Microbiology Vol. 13:1-15). A Cas9-gRNA complex recognizes a 3′ PAM sequence (NGG for the S. pyogenes Cas9) at the target site, permitting the spacer of the guide RNA to invade the double-stranded DNA target, and, if sufficient homology between the spacer and protospacer exists, generate a double-strand break cleavage. Cas9 endonucleases comprise RuvC and HNH domains that together produce double strand breaks, and separately can produce single strand breaks. For the S. pyogenes Cas9 endonuclease, the double-strand break leaves a blunt end. Cpf1 is a Clas 2 Type V Cas endonuclease, and comprises nuclease RuvC domain but lacks an HNH domain (Yamane et al., 2016, Cell 165:949-962). Cpf1 endonucleases create “sticky” overhang ends.

Some uses for Cas9-gRNA systems at a genomic target site include, but are not limited to, insertions, deletions, substitutions, or modifications of one or more nucleotides at the target site; modifying or replacing nucleotide sequences of interest (such as a regulatory elements); insertion of polynucleotides of interest; gene knock-out; gene-knock in; modification of splicing sites and/or introducing alternate splicing sites; modifications of nucleotide sequences encoding a protein of interest; amino acid and/or protein fusions; and gene silencing by expressing an inverted repeat into a gene of interest.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave . The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell. An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i) - (iii).

A “polynucleotide modification template” is also provided that comprises at least one nucleotide modification when compared to the nucleotide sequence to be edited. For example, a modification in the endogenous gene corresponding to SEQ ID NO: 1 to induce an amino substitution in the encoded polypeptide. A nucleotide modification can be at least one nucleotide substitution, addition, deletion, or chemical alteration. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In certain embodiments of the methods disclosed herein, a polynucleotide of interest is inserted at a target site and provided as part of a “donor DNA” molecule. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome. The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10: 957-963). The amount of homology or sequence identity shared by a target and a donor polynucleotide can vary and includes total lengths and/or regions.

The process for editing a genomic sequence at a Cas9-gRNA double-strand-break site with a modification template generally comprises: providing a host cell with a Cas9-gRNA complex that recognizes a target sequence in the genome of the host cell and is able to induce a double-strand-break in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the double-strand break. Genome editing using double-strand-break-inducing agents, such as Cas9-gRNA complexes, has been described, for example in US20150082478 published on 19 Mar. 2015, WO2015026886 published on 26 Feb. 2015, WO2016007347 published 14 Jan. 2016, and WO2016025131 published on 18 Feb. 2016.

To facilitate optimal expression and nuclear localization for eukaryotic cells, the gene comprising the Cas endonuclease may be optimized as described in WO2016186953 published 24 Nov. 2016, and then delivered into cells as DNA expression cassettes by methods known in the art. In certain embodiments, the Cas endonuclease is provided as a polypeptide. In certain embodiments, the Cas endonuclease is provided as a polynucleotide encoding a polypeptide. In certain embodiments, the guide RNA is provided as a DNA molecule encoding one or more RNA molecules. In certain embodiments, the guide RNA is provided as RNA or chemically modified RNA. In certain embodiments, the Cas endonuclease protein and guide RNA are provided as a ribonucleoprotein complex (RNP).

In certain embodiments of the inventive methods described herein the endogenous gene is modified by a zinc-finger-mediated genome editing process. The zinc-finger-mediated genome editing process for editing a chromosomal sequence includes for example: (a) introducing into a cell at least one nucleic acid encoding a zinc finger nuclease that recognizes a target sequence in the chromosomal sequence and is able to cleave a site in the chromosomal sequence, and, optionally, (i) at least one donor polynucleotide that includes a sequence for integration flanked by an upstream sequence and a downstream sequence that exhibit substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

A zinc finger nuclease includes a DNA binding domain (i.e., zinc finger) and a cleavage domain (i.e., nuclease). The nucleic acid encoding a zinc finger nuclease may include DNA or RNA. Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Choo et al. (2000) Curr. Opin. Struct. Biol. 10:411-416; and Doyon et al. (2008) Nat. Biotechnol. 26:702-708; Santiago et al. (2008) Proc. Natl. Acad. Sci. USA 105:5809-5814; Umov, et al., (2010) Nat Rev Genet. 11(9):636-46; and Shukla, et al., (2009) Nature 459 (7245):437-41. An engineered zinc finger binding domain may have a novel binding specificity compared to a naturally occurring zinc finger protein. As an example, the algorithm of described in U.S. Pat. No. 6,453,242 may be used to design a zinc finger binding domain to target a preselected sequence. Nondegenerate recognition code tables may also be used to design a zinc finger binding domain to target a specific sequence (Sera et al. (2002) Biochemistry 41:7074-7081). Tools for identifying potential target sites in DNA sequences and designing zinc finger binding domains may be used (Mandell et al. (2006) Nuc. Acid Res. 34:W516-W523; Sander et al. (2007) Nuc. Acid Res. 35:W599-W605).

An exemplary zinc finger DNA binding domain recognizes and binds a sequence having at least about 80% sequence identity with the desired target sequence. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

A zinc finger nuclease also includes a cleavage domain. The cleavage domain portion of the zinc finger nucleases may be obtained from any endonuclease or exonuclease. Non-limiting examples of endonucleases from which a cleavage domain may be derived include, but are not limited to, restriction endonucleases and homing endonucleases. See, for example, 2010-2011 Catalog, New England Biolabs, Beverly, Mass.; and Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). One or more of these enzymes (or functional fragments thereof) may be used as a source of cleavage domains.

In certain embodiments of the methods described herein the endogenous gene is modified by using “custom” meganucleases produced to modify plant genomes (see e.g., WO 2009/114321; Gao et al. (2010) Plant Journal 1:176-187). The term “meganuclease” generally refers to a naturally occurring homing endonuclease that binds double-stranded DNA at a recognition sequence that is greater than 12 base pairs and encompasses the corresponding intron insertion site. Naturally occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease.

Naturally occurring meganucleases, for example, from the LAGLIDADG family, have been used to effectively promote site-specific genome modification in plants, yeast, Drosophila, mammalian cells and mice. Engineered meganucleases such as, for example, LIG-34 meganucleases, which recognize and cut a 22 basepair DNA sequence found in the genome of Zea mays (maize) are known (see e.g., US 20110113509).

In certain embodiments of the methods described herein the endogenous gene is modified by using TAL endonucleases (TALEN). TAL (transcription activator-like) effectors from plant pathogenic Xanthomonas are important virulence factors that act as transcriptional activators in the plant cell nucleus, where they directly bind to DNA via a central domain of tandem repeats. A transcription activator-like (TAL) effector-DNA modifying enzymes (TALE or TALEN) are also used to engineer genetic changes. See e.g., US20110145940, Boch et al., (2009), Science 326(5959): 1509-12. Fusions of TAL effectors to the FokI nuclease provide TALENs that bind and cleave DNA at specific locations. Target specificity is determined by developing customized amino acid repeats in the TAL effectors.

In certain embodiments of the methods described herein the endogenous gene is modified by using base editing, such as an oligonucleobase-mediated system. In addition to the double-strand break inducing agents, site-specific base conversions can also be achieved to engineer one or more nucleotide changes to create one or more EMEs described herein into the genome. These include for example, a site-specific base edit mediated by a C•G to T•A or an A•T to G•C base editing deaminase enzymes (Gaudelli et al., Programmable base editing of A•T to G•C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. Catalytically dead dCas9 fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA.

Further provided are methods of plant breeding comprising crossing any of the soybean plants described herein with a second plant to produce a progeny seed comprising at least one modification described herein. In certain embodiments, a plant is produced from the progeny seed.

The following are examples of specific embodiments of some aspects of the invention. The examples are offered for illustrative purposes only and are not intended to limit the scope of the invention in any way.

### Example 1

This example demonstrates the expression and detection of modified glycinin1 variants in a cell free expression system

The expression level of Glycinin1 was measured in various parts of soybean. Glycinin1 was dominantly expressed in seeds 25 days after flowering (DAF) based on RNAseq expression profiling analysis (Table 2).

TABLE 2

Expression profiling of glycinin 1 (bold) and other putative glycinin family members in soybean.

| | young_ leaf | flower | one cm pod | pod shell 10DAF | pod shell 14DAF | seed 10DAF | seed 14DAF | seed 21DAF | seed 25DAF | seed 28DAF | seed 35DAF | seed 42DAF | root | nodule |
|---|---|---|---|---|---|---|---|---|---|---|---|---|---|---|
| Glyma03g32020 | 0 | 0 | 0 | 0 | 0 | 2 | 2 | 12 | 26498 | 72276 | 218636 | 210908 | 0 | 0 |
| Glyma03g32030 | 0 | 1 | 0 | 1 | 0 | 0 | 0 | 5 | 34953 | 87714 | 288053 | 298305 | 0 | 0 |
| Glyma10g04280 | 0 | 0 | 0 | 0 | 0 | 0 | 1 | 3 | 11410 | 29176 | 166962 | 249297 | 0 | 0 |
| Glyma13g18450 | 0 | 0 | 0 | 0 | 0 | 0 | 0 | 0 | 5591 | 15500 | 92982 | 130315 | 0 | 0 |
| Glyma19g34770 | 0 | 0 | 0 | 0 | 0 | 0 | 1 | 10 | 62 | 48 | 225 | 186 | 0 | 0 |
| Glyma19g34780 | 1 | 0 | 0 | 0 | 0 | 0 | 0 | 0 | 883 | 2263 | 10523 | 12595 | 2 | 0 |

Mature wide-type glycinin1 protein has 476 amino acids, including 4 Trp and 6 Met residues. To increase Met and Trp content in soybean, candidate amino acids in glycinin1 were substituted with Met and Trp. Glycinin proteins undergo multiple post-translational modification steps before forming a mature hexamer structure. Post-translational processing in vitro for modified seed storage proteins in the most commonly used E. coli expression system does not create post-translational modifications. Therefore, a plant-based tobacco cell-free protein synthesis system (U.S. Pat. 10,612,031) to overexpress wild-type and engineered glycinin1 variants was used.

Tobacco cells (Nicotiana tabacum L. cv. Bright Yellow 2, BY-2) were cultivated in shake flasks with Murashige-Skoog medium until they were harvested when the packed cell volume reached ~20%. BY-2 lysates were prepared as described previously (U.S. Pat. 10,612,031). DNA fragments containing wild-type or glycinin1 variants with signal peptide were cloned into the tobacco cell-free expression vector pDAB135006 using Gibson assembly (New England Biolabs, Ipswich, MA). The vector contains the T7 promoter for transcription and the tobacco mosaic virus 5′ omega leader sequence to enable translation in BY-2 lysate. The coupled transcription-translation reactions were carried out in 100 µl or 500 µl aliquots at 500 rpm for 40-45 h in 96-well or 48-well microtiter plates under 25° C. using a Kuhner ISF1-X shaker (Basel, Switzerland). BY-2 cell free reactions were centrifuged at 12,000 g for 15 min to separate soluble and insoluble fractions for further analysis.

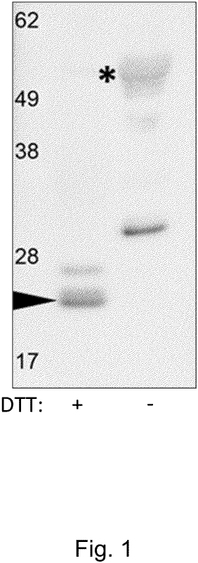

Protein solubility, stability and post-translational modification of glycinin1 variants was evaluated by Western blot using a novel polyclonal antibody targeting to the basic subunit of glycinin1 (SEQ ID 3). FIG. 1 shows that the pro-glycinin1 and its basic subunit were detected at expected molecular weight using this antibody when total seed protein from a soybean plant was extracted without or with DTT (to break down disulfide bonds).

### Example 2

This example demonstrates the generation of modified glycinin proteins

To generate modified glycinin proteins, amino acids in disordered regions of glycinin1 were targeted. The crystal structure of soybean proglycinin homotrimer (A1aB1b, PDB ID: 1FXZ) and homohexamer (A3B4, PDB ID: 1OD5) determined previously (Adachi et al., PNAS, 100, 7395-7400 (2003); Adachi et al. Journal of Molecular Biology, 305, 291-305 (2001)) was analyzed. The structure model shows proglycinin1 (A1aB1b) has five disordered regions, which are located at amino acid positions 20-28, 111-129, 195-214, 266-310 and 468-495, respectively. These flexible fragments are approximately correspondent to the variable regions based on sequence alignment with 11S globulins family members from legumes and non-legumes (Adachi et al. Journal of Molecular Biology, 305, 291-305 (2001)). Modified glycinins with 4-5 continuous Met insertions in the variable region were stable when overexpressed in E.coli and tobacco (Kim et al., Agricultural and Biological Chemistry, 54, 1543-1550 (1990); Takaiwa et al., Plant Science, 111, 39-49 (1995)). Therefore, to increase Met and Trp content in soybean, disordered regions of glycinin1 were targeted to insert or replace amino acid residues with Met or Trp. Table 3 shows total number of Met and Trp residues introduced to glycinin1 and their locations.

TABLE 3

List of glycinin1 variants with different number of Met or Trp residues that were introduced to the disordered region II to V, respectively.

| SEQ ID No. | Glycinin1 Variants | No. of Met Introduced | No. of Trp Introduced | Targeted Region | Amino Acid Changes |
|---|---|---|---|---|---|
| 4 | V9 | | 7 | disordered region IV | P300WWWWWWW |
| 5 | V11 | 6 | | disordered region II | Q118MMM Q119MMM |
| 6 | V12 | 5 | | disordered region III | Q196M Q197M E198M Q199M Q203M |
| 7 | V13 | 6 | | disordered region V | 486MMMMMM487 |
| 8 | V14 | 37 | | disordered region II, III, IV and V | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 |

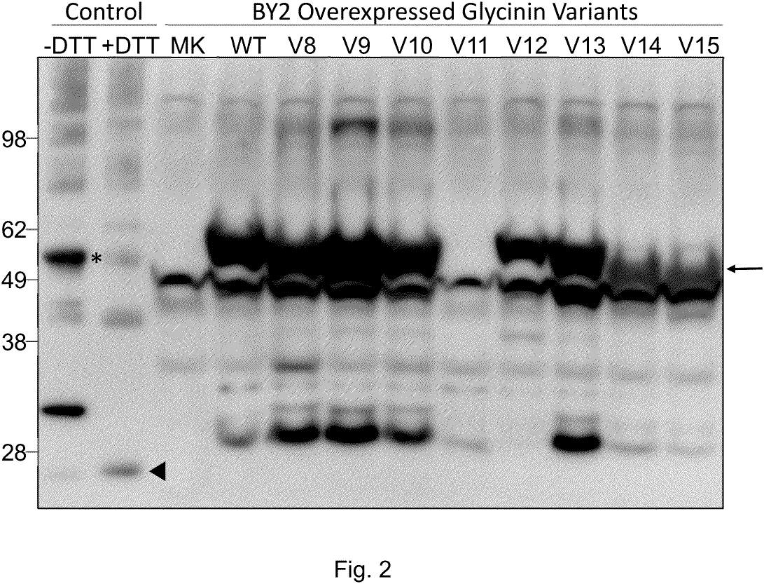

Protein expression vectors containing wild type glycinin1, glycinin1 variants or mock control (empty vector) were used for protein expression using the BY-2 cell free protein synthesis system as described in Example 1. Protein extract was processed for Western blot analysis using the anti-glycinin1 antibody. Interestingly, the majority of the wild type proglycinin1 was detected from the insoluble fraction containing organelles from BY-2 lysates, which suggests that glycinin 1 was targeted to microsomes in BY-2 lysate, where the signal peptide of glycinin1 was removed. As shown in FIG. 2, glycinin1 variants V9, V12, V13 and V14 with 7 Trp, 5 Met, 6 Met or 37 Met insertion, respectively, were expressed as the pro-glycinin1 and detected from the insoluble fractions. Surprisingly, variant V14 that contains a total of 37 Met insertion at all four targeted disorder regions was a stable protein, even though the variant V11 with 6 Met insertion only at disorder region II was not detected from Western blot. The glycinin1 variant V14 has 7 times more Met residues than wide type glycinin1. Although Trp is one of the largest amino acids, in variant V9 7 additional Trp residues were successfully inserted at disordered region IV, which almost triples Trp content compared to wide type glycinin1.

### Example 3

This example demonstrates the generation of modified glycinin proteins

To generate modified glycinin proteins, candidate amino acids for substitution were identified. In addition to targeting the disordered regions of glycinin1, candidate amino acid in glycinin1 were replaced based on the protein structure of the A1aB1b subunit and natural variation among 11S globulin family members. In particular, glycinin proteins consist of a few highly conserved sequence blocks connected by variable regions. Consistently, the conserved blocks correspond to the well-defined structure core, helix-hooks and jelly-roll, while the variable regions are often disordered or invisible in crystal structure. In addition, these variable regions are largely made of hydrophilic and/or charged residues, likely floating on the hexamer surface. The well-defined and conserved core residues are more suitable for Met or Trp replacement because both residues possess large size and high hydrophobicity with a natural tendency occupying protein’s interior.

Using structural information, the goal of the approach was to keep overall hydrophobicity largely unchanged and the protein surface intact, to minimize the impact on the protein packaging process. The sequence conservation and structural stereochemistry were also considered in selecting amino acid positions for replacement. For example, Tyr and Phe were considered as candidates for Trp replacement to accommodate its bulky size and aromatic structure. Leu was considered for Met substitution because of the linear side-chain and hydrophobicity of amino acid structure. To minimize the impact on packing procedures of glycinin1 during the post-translational modification process, Leu residues that are not directly involved in core packing were considered. Table 4 summarizes the number and location of Met and Trp residues introduced to glycinin1.

TABLE 4

List of glycinin1 variants with Met or Trp insertion to the regions that are conserved with other 11S globulin family members.

| SEQ ID No. | Glycinin1 Variants | No. of Met Introduced | No. of Trp Introduced | Targeted Region | AA changes |
|---|---|---|---|---|---|
| 9 | V8 | 13 | | conserved region | L39M L51M L74M L141M L175M L193M L226M L262M L321M L347M L376M L385M L390M |
| 10 | V10 | 13 | 7 | conserved region | L39M L51M L74M L141M L175M L193M L226M L262M L321M L347M L376M L385M L390M & Y104W Y134W Y153W Y334W F361W Y431W F480W |
| 11 | V15 | 37 | 7 | conserved region for Trp & disordered region II, III, IV and V for Met | Q118MMM Q119MMM Q196M Q197M E198M Q199M Q203M Q272M Q274M P276M E278M E280M E281M E282M E283M E284M D285M E286M P288M Q289M P300MMMMMMM 486MMMMMM487 & Y104W Y134W Y153W Y334W F361W Y431W F480W |

As illustrated in FIG. 2, the conserved region selected for Met and Trp replacement does not have significant impact on stability and solubility of the engineered glycinin1 variants. Variant V8 and V10, with 13 Met replacement and 13 Met plus 7 Trp substitutions respectively, both show similar protein expression level when compared with wild-type glycinin1. More importantly, variant V15 with 7 Trp replacement and 37 Met additions at 4 disordered regions was expressed as a stable pro-glycinin protein, although V15 migrates slightly faster than wild-type glycinin1 and other variants on SDS-page gel likely because the large number of Met and Trp replacements led to a higher isoelectric point for the V14 and V15 variants. Collectively, based on in vitro protein expression results, variant V15, the combination of replacing or insertion of Met and Trp residues at both conserved and disordered regions, showed similar protein stability to that of the wild-type proglycinin1 even after the number of Met and Trp residues was increased from 6 to 43 and from 4 to 11, respectively. These variants can significantly increase dry weight percentage of Met and Trp in soybean meal, which may reduce or eliminate the needs of synthetic alternatives for animal feeding.

### Example 4

This example demonstrates enhancing Met synthesis by removing the feedback sensitive self-regulatory domain of cystathionine-gamma synthase (CGS).

The content of Met and Cys, two sulfur containing amino acids, in soybean seeds is not high enough for animal nutrition. Transgenic soybean overexpressing Met rich protein, such as maize Zein, does not increase Met content in the plant (Koprivova et al., Journal of Genetics and Genomics, 43, 623-629 (2016)). Thus, besides using the “pull” strategy to increase sink Met content in glycinin1 as described in Examples 2 and 3, a “push” approach to enhance source Met content in soybean seeds to support production of engineered Met-rich storage proteins in seeds was also used.

There are two CGS genes in soybean, Glyma.09g235400 (GM-CGS1) and Glyma.18g261600 (GM-CGS2). Three gRNAs were designed to delete the self-regulatory domain of CGS (SEQ ID NO: 64) by Cas9/gRNA editing to generate a constantly active CGS1 or CGS2 in soybean. GM-CGS-gRNA1 (SEQ ID 22) and GM-CGS-gRNA2 (SEQ ID 23) were designed to dropout the self-regulatory domain in CGS1 protein by cutting after S41 and E130 of the CGS1 gene to remove the self-regulatory domain. GM-CGS-gRNA1 and GM-CGS-gRNA3 (SEQ ID 24) were used to dropout the self-regulatory domain in the CGS2 protein by cutting after S41 and E130 of the CGS2 gene to remove the self-regulatory domain.

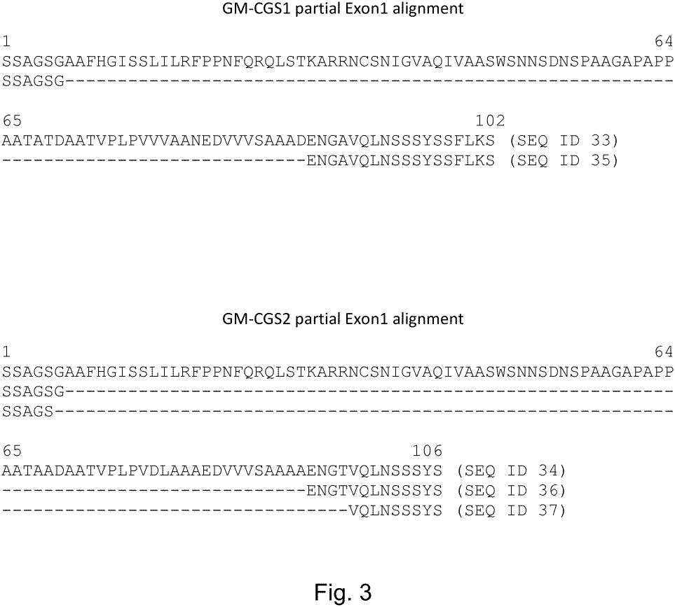

As shown in FIG. 3, several editing variants were created for CGS1 or CGS2 gene. For CGS1 gene. For the CGS1 gene, an editing variant with a 261 nucleotide in-frame deletion was created in exon 1, resulting in 87 amino acid deletion around the self-regulatory domain in the CGS1 protein. For the CGS2 gene, two variants were created with either a 261 nucleotide in-frame deletion (87 amino acid deletion) or a 276 nucleotide in-frame deletion (92 amino acid deletion) in exon 1.

By removing the self-feedback-inhibitory sequences in the CGS proteins, an increase methionine biosynthesis, increasing Met/Cys content in seeds and suppling more sulfur containing amino acids that are essential in animal nutrition is expected. Homozygous T2 seeds will be analyzed for sulfur amino acids and are expected to show increased amounts.

Single gRNA editing experiments were designed to introduce 3bp, 6bp, 9bp, 12bp, 15bp 18bp, 21bp or larger in-frame deletions in the self-regulatory domain region, resulting in smaller amino acid segment deletions in the CGS proteins. For example, the GM-CGS1-gRNA1 (SEQ ID 38) was designed in the exon1 of the GM-CGS1 gene to create a set of amino acid deletions near the peptide region of KARRNCSNIGVAQ inside the self-regulatory domain of the GM-CGS1 protein. In another example, the GM-CGS2-CR1 (SEQ ID 39) was also designed in the exon1 of the GM-CGS2 gene to create a set of amino acid deletions near the peptide region of KARRNCSNIGVAQ inside the self-regulatory domain of the GM-CGS2 protein. This editing strategy can create incremental amino acid deletions, from 1 amino acid deletion, up to 43 amino acid deletions for the full regulatory domain removal (amino acid position from #46 to #88 in soybean CGS1 (SEQ ID 43) or soybean CGS2 protein (SEQ ID 44)). Homozygous T2 seeds will be analyzed for sulfur amino acids and are expected to show increased amounts.

### Example 5

This example demonstrates increasing Met content in soybean by blocking the Methionine degradation pathway.

In addition to modulating the methionine biosynthetic pathway, the catabolic pathway of methionine was also identified as targets for gene editing. Methionine can be catabolized to 2-ketobutyrate by methionine beta-lyase (MGL). Two soybean MGL genes (glyma.02g087900 and glyma.13g001200) were identified as the editing targets for frameshift knockout. The GM-MGL-gRNA1 (SEQ ID 47) was designed to create frameshift knockout in the exon1 of the glyma.13g001200 (SEQ ID 45). The GM-MGL-gRNA2 (SEQ ID 48) was designed to create frameshift knockout in the exon1 of the glyma.13g001200 (SEQ ID 46). Two gRNA were introduced into soybean cells, and a bi-allelic knockout had been created in both MGL genes in the same plants. Amino acid analyses will be conducted in the homozygous T2 seeds for methionine content in seeds which is expected to be increased.

### Example 6

This example demonstrates increasing lysine content in soybean seed by removing feedback inhibition of the lysine biosynthetic pathway or by blocking the lysine degradation pathway.

Identification of the amino acid residues responsible for the feedback inhibition in the soybean DHPS protein will provide editing targets for the DHPS genes. The same gene editing experimental strategy as described in Example 4 for the CGS genes is applied for the DHPS genes in plants.

Two soybean LKR/SDH genes were identified as the targets for gene editing. The GM-LKR-gRNA1 (SEQ ID 49) and GM-LKR-gRNA2 (SEQ ID 50) were used to dropout the glyma.13g115500 (GM-LKR/SDH 1 gene). The GM-LKR-gRNA3 (SEQ ID 51) and GM-LKR-gRNA4 (SEQ ID 52) were used to dropout the glyma. 17g044300 (GM-LKR/SDH 2 gene). Gene edited soybean plants will be created. Homozygous dropout T2 seeds will be analyzed for seed amino acid composition improvement and are expected to show increases in the amount of lysine in the seed.

### Example 7

This example demonstrates increasing Met and Trp by deleting beta-congycinin and rebalancing the 7S and 11S proteome.

The Met and Trp contents of glycinin and β-conglycinin in conventional unmodified soybean seeds were calculated. Tables 5 and 6 summarize Met and Trp content among all isoforms of mature β-conglycinin (Table 5) and glycinin (Table 6). Across all isoforms, the average Met and Trp content (amino acid frequency) in β-conglycinin is 0.44% and 0.26%, respectively, compared to 1.15% and 0.83% of Met and Trp in glycinin.

TABLE 5

Comparison of Met and Trp content (% by amino acid frequency) among all isoforms of mature β-conglycinin

| β-conglycinin | α | α′ | β |
|---|---|---|---|
| Met | 0.69 | 0.18 | 0 |
| Trp | 0.34 | 0.18 | 0 |

TABLE 6

Comparison of Met and Trp content (% by amino acid frequency) among all isoforms of mature glycinin

| Glycinin | A1aB1b | A1bB2 | A2B1a | A3B4 | A5A4B3 |
|---|---|---|---|---|---|
| Met | 1.26 | 1.08 | 1.49 | 0.81 | 0.37 |
| Trp | 0.84 | 0.64 | 0.85 | 0.81 | 1.11 |

gRNAs were designed to knockout 6 putative β-conglycinin isoforms by Cas9/gRNA editing to rebalance the proteome to glycinin, which has higher Met and Trp content. 7 putative β-conglycinin candidates were identified including 3 α, 2 α′ and 2 β isoforms. Except for Glyma.10g246400 (α) and Glyma.20g146200 (β), all other isoforms show relatively high expression level at 30 or 50 days after flowering (DAF) in soybean seeds (Table 7).

TABLE 7

Expression level of 7 putative β-conglycinin isoforms in soybean seeds 30 or 50 days after flowering.

| | |
|---|---|
| β-conglycinin | Expression level measured by RNAseq |
| Glyma.20g148200 (β) SEQ ID NO: | 19251 (30DAF) |
| Glyma.20g148300 (α) | 67117 (30DAF) |
| Glyma.20g148400 (α) | 91647 (30DAF) |
| Glyma.20g146200 (β) | 7068 (30DAF) |
| Glyma.10g246300 (α′) | 86918 (30DAF) |
| Glyma.10g246500 (α′) | 20492 (50DAF) |
| Glyma.10g246400 (α) | No/low expression 6 (30DAF) |

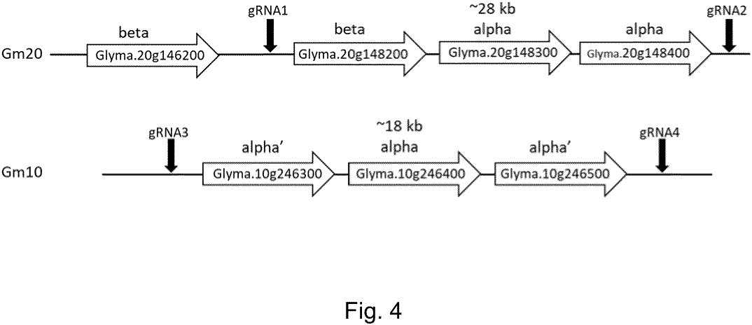

Four gRNAs were used to delete 6 of 7 β-conglycinin isoforms. The GM-CONG-gRNA1 (SEQ ID 25) and GM-CONG-gRNA2 (SEQ ID 26) was used to dropout the conglycinin cluster on chromosome 20 (Gm20); the GM-CONG-gRNA3 (SEQ ID 27) and GM-CONG-gRNA4 (SEQ ID 28) were used to dropout the conglycinin cluster on chromosome 10 (Gm10), as illustrated in FIG. 4. Due to the location of the glyma.20g146200 gene, this moderate-level expressed gene was not included in this dropout design.

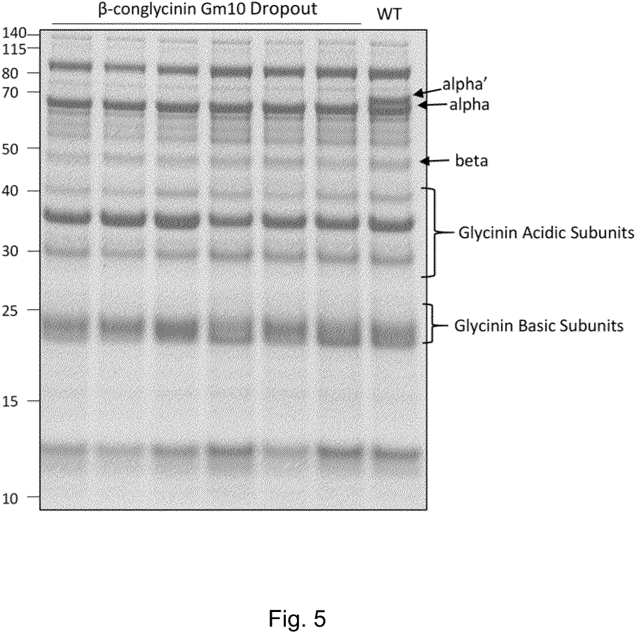

T2 homozygous seeds from the conglycinin Gm10 locus dropout experiment have been generated. Seed protein analyses were conducted by SDS-PAGE Coomassie Blue gel staining analyses (FIG. 5). No alpha’ subunits of conglycinin proteins can be detected in the T2 homozygous seeds from the Gm10 locus dropout variants, demonstrating complete removal of the conglycinin alpha’ subunit proteins in soybean seeds, in agreement with the complete removal of their genes from soybean genome. The total protein content of these T2 seeds did not change as compared to wild type seeds, indicating other soybean proteins are compensating for the loss of conglycinin alpha’ subunit proteins in these editing variants.

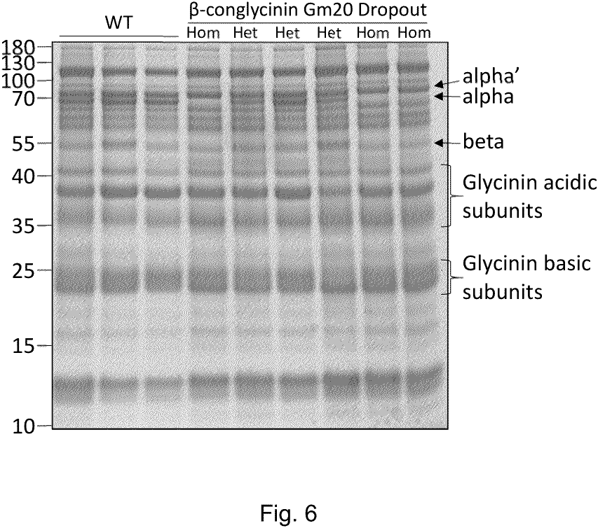

For the second editing experiment, the T2 seeds from the Gm20 locus dropout have also been analyzed by protein gel analyses (FIG. 6). The data indicate that the conglycinin alpha subunit proteins have been completely removed in the seeds of the homozygous dropout plants. The data also indicate that the conglycinin beta subunit protein was reduced in the dropout variant due to the elimination of Glyma.20g148200 gene. However, some of the beta subunits can be detected likely due to the dropout design not including the moderate expressed Glyma.20g146200 genes. The generated alpha’ and alpha/beta dropout loci will be genetically crossed together to create the complete conglycinin knockout soybean seeds. The amino acid composition of the soybean seeds with either single locus dropout or bi-loci dropout will be analyzed for amino acid improvement.

In another editing experiment, three gRNAs (SEQ ID 40, 41, 42) to do frameshift knockout of 5 highly expressed conglycinin genes (glyma.20g148200, glyma.20g148300, glyma.20g148400, glyma.10g246300 and glyma.10g246500), along with the moderate-level expressed glyma.20g146200, in a multiplex frameshift knockout approach were designed. The Homozygous T2 seeds will be analyzed for protein profile change and amino acid composition improvement.

Assuming seed protein content and amino acid composition of other seed storage proteins are not changed by protein rebalance, in the beta-conglycinin knockout soybean, glycinin content will be increased to 50% of seed protein, which should result in a 35% increase in Met+Cys, a 32% increase in Trp and a 29% increase in Thr (Table 8).

TABLE 8

Predicted Met, Cys, Trp, Thr, and Lys % change in seed protein after protein rebalance by knockout β-conglycinin

| | beta-conglycinin | Glycinin | WT 30% glycinin 20% beta-cong | Re-balance 50% glycinin 0% beta-cong | % change in protein |
|---|---|---|---|---|---|
| amino acid | % by WT | % by WT | | | |
| Met | 0.55 | 1.62 | 0.596 | 0.81 | 36% |
| Cys | 0.75 | 2.06 | 0.768 | 1.03 | 34% |
| Met+Cys | 1.3 | 3.68 | 1.364 | 1.84 | 35% |
| Trp | 0.5 | 1.26 | 0.478 | 0.63 | 32% |
| Thr | 1.61 | 3.69 | 1.429 | 1.845 | 29% |
| Lys | 6.48 | 5.66 | 2.994 | 2.83 | -5% |

### Example 8

This example demonstrates generating soybean plants with increased Met and Trp content in seeds by combinational approaches.

Beyond creating Met and Trp rich glycinin1 variants by protein engineering, 7S to 11S proteome rebalancing by deleting β-conglycinin subunits and creating constant active CGS by Cas9 editing individually as described above, three constructs to generate stably transformed soybean plants to stack the different approaches together were made. Specifically, a combined “pull” and “push” strategy by creating molecular stacks (Table 9) that overexpressing Met/Trp rich glycinin1 variants V10, V15 and V16 (SEQ ID 21), de-regulated CGS (SEQ ID 29) and a RNAi cassette (SEQ ID 30) to knock down 6 isoforms of β conglycinin subunits was generated. Soybean seeds harvested from the transformed plants can be used to measure total protein content and amino acid composition.

TABLE 9

Transgenic constructs that overexpressing Met/Trp rich glycinin1 variants, de-regulated CGS and a RNAi cassette to knock down 6 isoforms of β-conglycinins in soybean.
Genes

| Construct | Glycinin1 variants | De-regulated CGS variant | β-conglycinin RNAi |
|---|---|---|---|
| 1 | GmGY1 PRO::Glycinin_V10::GmGY1 TERM | AT-UBIQ10 PRO::GM-deCGS::PHASEOLIN TERM | GM-KTI3 PRO::GM-βCON_RNAi::UBQ14 TERM |
| 2 | GmGY1 PRO::Glycinin_V15::GmGY1 TERM | AT-UBIQ10 PRO::GM-deCGS::PHASEOLIN TERM | GM-KTI3 PRO::GM-βCON_RNAi::UBQ14 TERM |
| 3 | GmGY1 PRO::Glycinin_V16::GmGY1 TERM | | |

The Met/Trp rich glycine1 variants can also be created using gene editing at the native glycine gene loci with homology-dependent repair process, by proving a donor DNA with the desired V10, V15 and V16 glycine1 variants as a repair template. Furthermore, similar to the transgenic strategy as outlined by molecular stack constructs, these edited glycinin1 variants can be genetically crossed with gene editing lines, such as de-regulated CGS1/CGS2 lines, or MGL lines, or de-regulated DHPS lines, or LKR/SDK knockout lines (Example 4, 5, 6) and β-conglycinin knockout line (Example 7) to increase the production of Met, Lys as well as to rebalance proteome to glycinins, which is expected to significantly increase Met/Trp/Lys content in seeds.

### Example 9

This example demonstrates generating soybean plants with decreased raffinose family of oligosaccharides (RFO) content in seeds by raffinose synthase gene knockouts.

Raffinose and stachyose, members of the raffinose family of oligosaccharides (RFO), are two of the major insoluble carbohydrate components in soybean seeds. There are several raffinose synthase (RS) genes in soybean, including the RS2 (glyma.06g179200), RS3 (glyma.05g003900) and RS4 (glyma.05g040300). A pair of gRNAs, GM-RS2-gRNA1 (SEQ ID. 65) and GM-RS2-gRNA2 (SEQ ID. 66) were designed to dropout the RS2 gene. Another pair of gRNAs, GM-RS4-gRNA1 (SEQ ID. 67) and GM-RS4-gRNA2 (SEQ ID. 68) were designed to dropout the RS4 gene.

Dropout variants have been identified in T0 soybean plants. The raffinose and stachyose content in soybean seeds will be analyzed in homozygous dropout T2 seeds.

gRNAs were also designed to create frameshift mutations in the RS2, RS3 and RS4 genes. The GM-RS2-gRNA3 (SEQ ID. 69) was designed to target the exon1 on the RS2 gene; the GM-RS3-gRNA1 (SEQ ID.70) was designed to target the exon1 on the RS3 gene; and the GM-RS4-gRNA3 (SEQ ID.71) was designed to target the exon1 on the RS4 gene. These gRNAs were introduced into soybean cells either individually, or in different combinations. The raffinose and stachyose content in gene edited soybean seeds will be analyzed in homozygous T1 or T2 seeds.

### Example 10

This example demonstrates generating soybean plants with increased protein content in seeds.

Protein is the most valuable component in soybean meal. Soybean varieties with increased seed protein content were developed by integrating high protein QTLs from a high protein donor line such as Danbaekkong (Prenger et al. 2019 Crop Sci. 59:2498-2508). Additionally, from a fast neutron mutagenized population, 7 high protein mutants were isolated by phenotypic screening (Bolon et al 2011, Plant Physiology 156:240-253). The highest protein mutant, L10, contains 58.0% seed protein on dry weight basis (Islam et al. 2019 BMC Plant Biology 19:420). Soybean plants expressing the Arabidopsis QQS gene also have increased protein content in seed (Li et al. 2015 PNAS 1112: 14734-14739).

To identify additional high protein mutants without a reduction of oil, or with an increase in oil, approximately 10,000 soybean seeds were irradiated at dose of 100 Gy, 200 Gy and 300 Gy using ⁶⁰Co gamma radiation at the Radiation Science and Engineering Center (RSEC) of The Pennsylvania State University. Treated M1 seeds were planted in field to generate M2 seeds. M2 seeds on M1 plants were harvested by a combine in bulk and used for single seed protein and oil sorting on a high throughput seed sorter (Q-Sorter Explorer manufactured by QualySense Ag, Glattbrugg, Switzerland) using near-infrared detection to assess the composition of each seed. Approximately 160 lb M2 seeds were run through a qSorter to identify high protein and oil seeds. After sorting for both protein and oil, approximately 5000 high protein and oil M2 seeds were identified and planted in a field at 50 seeds per short row. Plants were pulled and threshed individually. Approximately 3800 M2 plants set more than 50 seeds. Protein and oil content of all seeds from a single M2 plant were determined by an automated FT-NIR spectroscopy machine as described previously (Roesler et al. An Improved Variant of Soybean Type 1 Diacylglycerol Acyltransferase Increases the Oil Content and Decreases the Soluble Carbohydrate Content of Soybeans. Plant Physiol. 2016 171:878-93). Based on seed protein and oil data, the 50 top M2 mutant plants were selected from the 3800 M2 plants and these were advanced for M3 validation. M3 seeds from the top 50 M2 mutant plants were grown out in a single short row in the field. Early vigor and stand count data, and maturity date were collected. At maturity, all plants in the short row were pulled and threshed individually. Seed oil and protein content from each individual M3 plant were determined by FT-NIR. Based on agronomic performance and seed composition data, 29 M3 mutants were selected to advance to M4 multiple row tests to create M4 sublines. About 20 plants from a single M3 mutant short row were selected based on seed protein and oil content. Each plant become a subline of the mutant and grown out at a single row. Based on agronomic performance and seed composition data of sublines, 20 high protein mutants were validated in M4 generation (Table 10).

TABLE 10

Seed oil and protein content of 20 mutants

| | NIT seed oil % | NIT seed protein % |
|---|---|---|
| WT | 18.9 | 35.0 |
| Mutant #1 | 18.9 | 36.4 |
| Mutant #2 | 19.2 | 36.1 |
| Mutant #3 | 18.9 | 36.3 |
| Mutant #4 | 19.0 | 36.1 |
| Mutant #5 | 19.0 | 36.1 |
| Mutant #6 | 19.1 | 35.9 |
| Mutant #7 | 19.1 | 35.9 |
| Mutant #8 | 19.1 | 35.8 |
| Mutant #9 | 19.0 | 36.1 |
| Mutant #10 | 18.9 | 36.0 |
| Mutant #11 | 19.2 | 35.9 |
| Mutant #12 | 18.7 | 36.6 |
| Mutant #13 | 18.8 | 36.2 |
| Mutant #14 | 19.1 | 36.1 |
| Mutant #15 | 18.7 | 36.6 |
| Mutant #16 | 18.9 | 36.3 |
| Mutant #17 | 19.1 | 36.6 |
| Mutant #18 | 18.8 | 36.8 |
| Mutant #19 | 21.5 | 35.5 |
| Mutant #20 | 19.4 | 35.4 |

### Example 11

This example demonstrates trait stacking to increase essential amino acid content in seeds.

Soybean meal is the by-product of the extraction of soybean oil. Meal protein content can be increased by increasing seed protein content, and a higher protein content in meal should result in an increase the amount of available essential amino acids, such as methionine, lysine, threonine and tryptophan.

To further increase the essential amino acid content in soybean one or more of the modifications described in Examples 1-8 (e.g., modification of the glycinin protein; knockout of at least one beta-conglycinin isoform; increasing CGS activity; decreasing the expression and/or activity of MGL; decreasing the expression and/or activity of LKR/SDH; and a modification increasing the activity of DHPS) will be combined (e.g., stacked) with a modification that increases total protein content, such as those described in Example 10. The stack will be produced by crossing high protein mutants with high amino acid variants (Table 11). The combination of increased protein modification(s) with increased essential amino acid modification(s) in seeds should result in a further increase the availability of essential amino acids, such as Met, Lys, Thr, and Trp in the meal which should meet animal nutritional needs.

To further improve meal quality, one or more of the modifications described in Examples 1-8 or Example 10 will be combined with one or more of the modifications described in Example 9 (e.g., knockout of a raffinose synthase gene to decrease RFO content). The combination will be produced by crossing low RFO variants to either high protein mutants or high amino acid variants (Table 11). The combination of blocking RFO synthesis should result in more sucrose available for oil and protein biosynthesis, improve protein digestibility, and an increase in available energy and amino acid for animal growth.

A stack of all three traits (e.g., increased essential amino acid; increased total protein, decreased RFO) will be produced by crossing a low RFO soybean with an increased protein and increased essential amino acid soybean. Soybean meal produced from grain with the stack of all three traits should result in enhanced meal quality (e.g., more nutritious and more available energy).

TABLE 11

List of stacks of high protein and high essential amino acids and reduced RFO by crossing

| | | |
|---|---|---|
| High protein | High essential amino acid | Low RFO |
| Fast neutron high protein mutant | High Met CGS variant | Raffinose synthase 2 (RS2) knockout variant |
| High protein CRISPR variant | High Met MGL variant | Raffinose synthase 3 (RS3) knockout variant |
| High protein transgenic event | High Lys LKR/SDH variant | Raffinose synthase 4 (RS4) knockout variant |
| Gamma-ray high protein mutant | High Met and Trp glycinin edited variant | RS2/RS3 knockout stack |
| | High Lys DHPS variant | RS2/RS4 knockout stack |
| | High Met, Thr, and Trp beta- conglycinin knockout variant | RS2/RS3/RS4 knockout stack |

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1-27. (canceled)

28. A soybean seed or a soybean plant producing a soybean seed comprising at least about a 10% increase in one or more essential amino acids, as compared to a control seed, wherein the soybean seed or soybean plant comprises a modification selected from the group consisting of (i) a targeted genetic modification of a gene encoding a glycinin protein to produce a modified glycinin protein comprising an insertion or substitution of one or more methionine, threonine, tryptophan, or lysine residues, or any combination thereof, (ii) a modification decreasing the expression of beta-conglycinin, (iii) a modification increasing the expression of a polynucleotide encoding cystathionine-gamma-synthase (CGS), (iv) a modification decreasing the expression and/or activity of methionine beta-lyase (MGL), (v) a modification decreasing the expression and/or activity of LKR/SDH, (vi) a modification increasing the expression or activity of dihydrodipicolinate synthase (DHPS), and (vii) any combination thereof.

29. The soybean seed or soybean plant of claim 28, wherein the soybean seed or the soybean plant comprises:
(a) a targeted genetic modification of a glycinin protein and a decrease in the expression of beta-conglycinin_{;}
(b) a targeted genetic modification of a glycinin protein and a modification of the polynucleotide encoding CGS;
(c) a decrease in the expression of beta-conglycinin and a modification of the polynucleotide encoding CGS; or
(d) a targeted genetic modification of a glycinin protein, a decrease in the expression of beta-conglycinin, and a modification of the polynucleotide encoding CGS.

30-33. (canceled)

34. The soybean seed or soybean plant of claim 29, wherein the soybean plant or the soybean seed of (a), (b), (c), or (d) further comprises a decrease in the expression and/or activity of MGL.

35-51. (canceled)

52. The soybean seed or soybean plant of claim 28, wherein the soybean seed or soybean plant comprises the modification decreasing the expression of beta-conglycinin, and the modification comprises a knockout of at least one gene encoding an isoform of beta-conglycinin comprising an amino acid sequence that is at least 90% identical to any one of SEQ ID NOs: 57-63.

53. (canceled)

54. The soybean plant or soybean seed of claim 28, wherein the ratio of glycinin to conglycinin in the soybean plant or soybean seed is at least 4:1.

55. The soybean seed or soybean plant of claim 28, wherein the soybean seed or soybean plant comprises the modification increasing the activity of the CGS gene, and the modification comprises a targeted genetic modification that removes a self-regulatory domain of a CGS gene, wherein the self-regulatory domain of the CGS gene encodes a polypeptide comprising an amino acid sequence that is at least 90% identical to SEQ ID NO: 64.

56. The soybean seed or soybean plant of claim 55, wherein the modified CGS gene encodes a CGS protein comprising an amino acid sequence that is at least 90% identical to any one of SEQ ID NOs: 35-37.

57. The soybean seed or soybean plant of claim 28, wherein the soybean seed or soybean plant comprises the modification decreasing the expression and/or activity of MGL, and the modification comprises a knockout of the MGL gene, wherein the MGL gene encodes an MGL protein comprising an amino acid sequence that is at least 90% identical to SEQ ID NO: 53 or 54.

58-59. (canceled)

60. The soybean seed or soybean plant of claim 28, wherein the seed produced from the soybean plant or the soybean seed comprises at least a 15% increase in the amount of methionine as compared to a control seed.

61-64. (canceled)

65. A soybean plant produced by the soybean seed of claim 28, wherein the soybean plant comprises the at least one modification.

66. A method of plant breeding comprising crossing the soybean plant of claim 28 with a second soybean plant to produce a progeny seed.

67-109. (canceled)

110. A soy protein composition comprising an essential amino acid content of at least 50% of the total amino acid content and at least one characteristic selected from the group consisting of: (a) a modified glycinin protein comprising at least a 10% increase in the proportion of methionine residues, tryptophan residues or a combination thereof of the total glycinin amino acid residues compared with a control glycinin protein; (b) the composition comprises less than 5% beta-conglycinin; (c) a CGS protein comprising an amino acid sequence that is at least 75% identical to any one of SEQ ID NOs: 35-37; (d) the composition lacks MGL; (e) the composition lacks LKR/SDH; (f) a modified DHPS protein; and (g) the composition comprises less than 5% RFO.

111-112. (canceled)

113. The soy protein composition of claim 110, wherein the composition lacks at least one beta-conglycinin isoform selected from the group consisting of SEQ ID NOs: 57-63.

114. (canceled)

115. The soy protein composition of claim 110, wherein the ratio of glycinin to beta conglycinin in the composition is at least 4:1.

116-123. (canceled)

124. A soy protein composition produced from the seeds of claim 28.

125-126. (canceled)

127. The soy protein composition of claim 110, wherein the composition is an animal feed or a human food.

128-136. (canceled)

137. A soybean seed or a soybean plant producing a soybean seed comprising at least about a 20% increase in methionine on a dry weight of seed basis and at least about a 2 percentage point increase in total protein measured on a dry weight basis, as compared to a control seed.

138. The soybean seed or the soybean plant of claim 137, wherein the soybean seed or soybean plant comprises a modification selected from the group consisting of (i) a targeted genetic modification of a gene encoding a glycinin protein to produce a modified glycinin protein comprising an insertion or substitution of one or more methionine, threonine, tryptophan, or lysine residues, or any combination thereof, (ii) a modification decreasing the expression of beta-conglycinin, (iii) a modification increasing the expression of a polynucleotide encoding cystathionine-gamma-synthase (CGS), (iv) a modification decreasing the expression and/or activity of methionine beta-lyase (MGL), (v) a modification decreasing the expression and/or activity of LKR/SDH, (vi) a modification increasing the expression or activity of dihydrodipicolinate synthase (DHPS), and (vii) any combination thereof.

139-148. (canceled)

149. The soybean seed or the soybean plant of claim 138, wherein the soybean seed or soybean plant comprises the modification decreasing the expression of beta-conglycinin and wherein the modification comprises a knockout of at least one gene encoding an isoform of beta-conglycinin comprising an amino acid sequence that is at least 90% identical to any one of SEQ ID NOs: 57-63.

150. (canceled)

151. The soybean seed or the soybean plant of claim 138, wherein the soybean seed or soybean plant comprises the modification increasing the activity of the CGS gene, wherein the modification comprises a targeted genetic modification that removes a self-regulatory domain of a the CGS gene, the self-regulatory domain encoding a polypeptide comprising an amino acid sequence that is at least 90% identical to SEQ ID NO: 64.

152. The soybean seed or soybean plant of claim 151, wherein the modified CGS gene encodes a CGS protein comprising an amino acid sequence that is at least 90% identical to any one of SEQ ID NOs: 35-37.

153. The soybean seed or soybean plant of claim 138, wherein the soybean seed or soybean plant comprises the modification decreasing the expression and/or activity of MGL, and wherein the modification comprises a knockout of the MGL gene, wherein the MGL gene encodes an MGL protein comprising an amino acid sequence that is at least 90% identical to SEQ ID NO: 53 or 54.

154-155. (canceled)

156. The soybean seed or soybean plant claim 137, wherein the ratio of glycinin to conglycinin in the soybean plant or soybean seed is at least 4:1.

157. A soybean plant produced by the soybean seed of claim 137, wherein the soybean plant comprises the at least one modification.

158. A method of plant breeding comprising crossing the soybean plant of claim 137 with a second soybean plant to produce a progeny seed.

159. The soybean seed or soybean plant of claim 137, wherein the soybean seed or soybean plant further comprises (a) a modification decreasing raffinose family oligosaccharides (RFO) content selected from the group consisting of a decrease in the expression or activity of raffinose synthase 2 (RS2), a decrease in the expression and/or activity of raffinose synthase 4 (RS4), and a decrease in expression and/or activity of both RS2 and RS4 or (b) a modification that increases the total protein content in the seed, as compared to a control seed or both (a) and (b).

160-163. (canceled)

164. The soybean seed or soybean plant of claim 28, wherein the soybean seed or soybean plant further comprises a modification decreasing raffinose family oligosaccharides (RFO) content selected from the group consisting of a decrease in the expression or activity of raffinose synthase 2 (RS2), a decrease in the expression and/or activity of raffinose synthase 4 (RS4), and a decrease in expression and/or activity of both RS2 and RS4 or (b) a modification that increases the total protein content in the seed, as compared to a control seed or both (a) and (b).

165-171. (canceled)


SEQUENCE LISTING (71 sequences)

SEQ ID NO: 1 - protein, 495 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 2 - DNA, 1488 bp, Glycine max
atggccaagctagttttttccctttgttttctgcttttcagtggctgctgcttcgctttcagttccagagagcagcctcagcaaaacgagtgccagatccaaaaactcaatgccatgaaaccggataaccgtatagagtcagaaggagggatgattgagacatggaaccctaacaacaagccattccagtgtgccggtgttgccctctctcgctgcaccatgaaccgcaacgcccttcgtagaccttcctacaccaacggtccccaggaaatctacatccaacaaggtaagggtatttttggcatgatatggccgggttgtcctagcacatttgaagagcctcaacaacctcaacaaagaggacaaagcagcagaccacaagaccgtcaccagaagatctggaacttcagagagggtgatatgatcgcagtgcctactggtgttgcatggtggatgtggaacaatgaagacactcctgttgttgccgtttctattattgacaccaacagcttggagaaccagatggaccagatgcctaggagattctatcttgctgggaaccaagagcaagagtttatgaaatatcagcaagagcaaggaggtcatcaaagccagaaaggaaagcatcagcaagaagaagaaaacgaaggaggcagcatattgagtggcttcaccatggaattcttggaacatgcattcagcgtggacaagcagatagcgaaaaacctacaaggagagaacgaaggggaagacaagggagccattgtgacagtgaaaggaggtatgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagaatgcgccacaacattggccagacttcatcacctgacatctggaaccctcaagccggtagcgtcacaaccgccaccagcatggacttcccagccctctcgtggctcagactcagtgctgagtggggatctctccgcaagaatgcaatgttcgtgccacactacaacatgaacgcgaacagcataatatacgcaatgaatggacgggcaatgatacaagtggtgaattgcaacggtgagagagtgtttgatggagagctgcaagagggacgggtgctgatcgtgccacaaaactttgtggtggctgcaagatcacagagtgacaacttcgagtgggtgtcattcaagaccaatgatacacccatgatcggcactcttgcaggggcaaactcattgttgaacgcattaccagaggaagtgattcagcacactttcaacctaaaaagccagcaggccaggcagataaagaacaacaacccttggaagttcctggttccacctcaggagtctcagaagagagctgtggcttag

SEQ ID NO: 3 - protein, 14 aa, Glycine max
CNGERVFDGELQEG

SEQ ID NO: 4 - protein, 501 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRWWWWWWWRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 5 - protein, 499 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPMMMMMMRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 6 - protein, 495 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYMMMMGGHMSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 7 - protein, 501 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPMMMMMMQESQKRAVA

SEQ ID NO: 8 - protein, 511 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPMMMMMMRGQSSRPQDRHQKIYNFREGDLIAVPTGVAWWMYNNEDTPVVAVSIIDTNSLENQLDQMPRRFYLAGNQEQEFLKYMMMMGGHMSQKGKHQQEEENEGGSILSGFTLEFLEHAFSVDKQIAKNLQGENEGEDKGAIVTVKGGLSVIKPPTDEMQMRMQMEMMMMMMMKMMCKGKDKHCQRMMMMMMMRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPMMMMMMQESQKRAVA

SEQ ID NO: 9 - protein, 495 aa, Glycine max
MAKLVFSLCFLLFSGCCFAFSSREQPQQNECQIQKLNAMKPDNRIESEGGMIETWNPNNKPFQCAGVALSRCTMNRNALRRPSYTNGPQEIYIQQGKGIFGMIYPGCPSTFEEPQQPQQRGQSSRPQDRHQKIYNFREGDMIAVPTGVAWWMYNNEDTPVVAVSIIDTNSLENQMDQMPRRFYLAGNQEQEFMKYQQEQGGHQSQKGKHQQEEENEGGSILSGFTMEFLEHAFSVDKQIAKNLQGENEGEDKGAIVTVKGGMSVIKPPTDEQQQRPQEEEEEEEDEKPQCKGKDKHCQRPRGSQSKSRRNGIDETICTMRMRHNIGQTSSPDIYNPQAGSVTTATSMDFPALSWLRLSAEFGSLRKNAMFVPHYNMNANSIIYAMNGRAMIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 10 - protein, 495 aa, Glycine max
MAKLVFSLCFLLFSGCCFAFSSREQPQQNECQIQKLNAMKPDNRIESEGGMIETWNPNNKPFQCAGVALSRCTMNRNALRRPSYTNGPQEIYIQQGKGIFGMIWPGCPSTFEEPQQPQQRGQSSRPQDRHQKIWNFREGDMIAVPTGVAWWMWNNEDTPVVAVSIIDTNSLENQMDQMPRRFYLAGNQEQEFMKYQQEQGGHQSQKGKHQQEEENEGGSILSGFTMEFLEHAFSVDKQIAKNLQGENEGEDKGAIVTVKGGMSVIKPPTDEQQQRPQEEEEEEEDEKPQCKGKDKHCQRPRGSQSKSRRNGIDETICTMRMRHNIGQTSSPDIWNPQAGSVTTATSMDFPALSWLRLSAEWGSLRKNAMFVPHYNMNANSIIYAMNGRAMIQVVNCNGERVFDGELQEGRVLIVPQNFVVAARSQSDNFEWVSFKTNDTPMIGTLAGANSLLNALPEEVIQHTFNLKSQQARQIKNNNPWKFLVPPQESQKRAVA

SEQ ID NO: 11 - protein, 511 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIWPGCPSTFEEPQQPMMMMMMRGQSSRPQDRHQKIWNFREGDLIAVPTGVAWWMWNNEDTPVVAVSIIDTNSLENQLDQMPRRFYLAGNQEQEFLKYMMMMGGHMSQKGKHQQEEENEGGSILSGFTLEFLEHAFSVDKQIAKNLQGENEGEDKGAIVTVKGGLSVIKPPTDEMQMRMQMEMMMMMMMKMMCKGKDKHCQRMMMMMMMRGSQSKSRRNGIDETICTMRLRHNIGQTSSPDIWNPQAGSVTTATSLDFPALSWLRLSAEWGSLRKNAMFVPHYNLNANSIIYALNGRALIQVVNCNGERVFDGELQEGRVLIVPQNFVVAARSQSDNFEWVSFKTNDTPMIGTLAGANSLLNALPEEVIQHTFNLKSQQARQIKNNNPWKFLVPPMMMMMMQESQKRAVA

SEQ ID NO: 12 - protein, 511 aa, Glycine max
MAKLVFSLCFLLFSGCCFAFSSREQPQQNECQIQKLNAMKPDNRIESEGGMIETWNPNNKPFQCAGVALSRCTMNRNALRRPSYTNGPQEIYIQQGKGIFGMIWPGCPSTFEEPQQPMMMMMMRGQSSRPQDRHQKIWNFREGDMIAVPTGVAWWMWNNEDTPVVAVSIIDTNSLENQMDQMPRRFYLAGNQEQEFMKYMMMMGGHMSQKGKHQQEEENEGGSILSGFTMEFLEHAFSVDKQIAKNLQGENEGEDKGAIVTVKGGMSVIKPPTDEQQQRPQEEEEEEEDEKPQCKGKDKHCQRMMMMMMMRGSQSKSRRNGIDETICTMRMRHNIGQTSSPDIWNPQAGSVTTATSMDFPALSWLRLSAEWGSLRKNAMFVPHYNMNANSIIYAMNGRAMIQVVNCNGERVFDGELQEGRVLIVPQNFVVAARSQSDNFEWVSFKTNDTPMIGTLAGANSLLNALPEEVIQHTFNLKSQQARQIKNNNPWKFLVPPMMMMMMQESQKRAVA

SEQ ID NO: 13 - DNA, 1506 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctcaacaaagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatcagcaagagcaaggaggtcatcaaagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgctggtggtggtggtggtggtggcgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttag

SEQ ID NO: 14 - DNA, 1500 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctatgatgatgatgatgatgagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatcagcaagagcaaggaggtcatcaaagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttag

SEQ ID NO: 15 - DNA, 1488 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctcaacaaagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatatgatgatgatgggaggtcatatgagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttag

SEQ ID NO: 16 - DNA, 1506 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctcaacaaagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatcagcaagagcaaggaggtcatcaaagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctatgatgatgatgatgatgcaggagtctcagaagagagctgtggcttag

SEQ ID NO: 17 - DNA, 1536 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctatgatgatgatgatgatgagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatatgatgatgatgggaggtcatatgagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagatgcaaatgagaatgcagatggagatgatgatgatgatgatgatgaagatgatgtgcaagggtaaagacaaacactgccaacgcatgatgatgatgatgatgatgcgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctatgatgatgatgatgatgcaggagtctcagaagagagctgtggcttag

SEQ ID NO: 18 - DNA, 1488 bp, Glycine max
atggccaagctagttttttccctttgttttctgcttttcagtggctgctgcttcgctttcagttccagagagcagcctcagcaaaacgagtgccagatccaaaaactcaatgccatgaaaccggataaccgtatagagtcagaaggagggatgattgagacatggaaccctaacaacaagccattccagtgtgccggtgttgccctctctcgctgcaccatgaaccgcaacgcccttcgtagaccttcctacaccaacggtccccaggaaatctacatccaacaaggtaagggtatttttggcatgatatacccgggttgtcctagcacatttgaagagcctcaacaacctcaacaaagaggacaaagcagcagaccacaagaccgtcaccagaagatctataacttcagagagggtgatatgatcgcagtgcctactggtgttgcatggtggatgtacaacaatgaagacactcctgttgttgccgtttctattattgacaccaacagcttggagaaccagatggaccagatgcctaggagattctatcttgctgggaaccaagagcaagagtttatgaaatatcagcaagagcaaggaggtcatcaaagccagaaaggaaagcatcagcaagaagaagaaaacgaaggaggcagcatattgagtggcttcaccatggaattcttggaacatgcattcagcgtggacaagcagatagcgaaaaacctacaaggagagaacgaaggggaagacaagggagccattgtgacagtgaaaggaggtatgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagaatgcgccacaacattggccagacttcatcacctgacatctacaaccctcaagccggtagcgtcacaaccgccaccagcatggacttcccagccctctcgtggctcagactcagtgctgagtttggatctctccgcaagaatgcaatgttcgtgccacactacaacatgaacgcgaacagcataatatacgcaatgaatggacgggcaatgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttag

SEQ ID NO: 19 - DNA, 1488 bp, Glycine max; identical to SEQ ID NO: 2

SEQ ID NO: 20 - DNA, 1536 bp, Glycine max
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatggccgggttgtcctagcacatttgaagagcctcaacaacctatgatgatgatgatgatgagaggacaaagcagcagaccacaagaccgtcaccagaagatctggaacttcagagagggtgatttgatcgcagtgcctactggtgttgcatggtggatgtggaacaatgaagacactcctgttgttgccgtttctattattgacaccaacagcttggagaaccagctcgaccagatgcctaggagattctatcttgctgggaaccaagagcaagagtttctaaaatatatgatgatgatgggaggtcatatgagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagatgcaaatgagaatgcagatggagatgatgatgatgatgatgatgaagatgatgtgcaagggtaaagacaaacactgccaacgcatgatgatgatgatgatgatgcgaggaagccaaagcaaaagcagaagaaatggcattgacgagaccatatgcaccatgagacttcgccacaacattggccagacttcatcacctgacatctggaaccctcaagccggtagcgtcacaaccgccaccagccttgacttcccagccctctcgtggctcagactcagtgctgagtggggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtgggtgtcattcaagaccaatgatacacccatgatcggcactcttgcaggggcaaactcattgttgaacgcattaccagaggaagtgattcagcacactttcaacctaaaaagccagcaggccaggcagataaagaacaacaacccttggaagttcctggttccacctatgatgatgatgatgatgcaggagtctcagaagagagctgtggcttag

SEQ ID NO: 21 - DNA, 1536 bp, Glycine max
atggccaagctagttttttccctttgttttctgcttttcagtggctgctgcttcgctttcagttccagagagcagcctcagcaaaacgagtgccagatccaaaaactcaatgccatgaaaccggataaccgtatagagtcagaaggagggatgattgagacatggaaccctaacaacaagccattccagtgtgccggtgttgccctctctcgctgcaccatgaaccgcaacgcccttcgtagaccttcctacaccaacggtccccaggaaatctacatccaacaaggtaagggtatttttggcatgatatggccgggttgtcctagcacatttgaagagcctcaacaacctatgatgatgatgatgatgagaggacaaagcagcagaccacaagaccgtcaccagaagatctggaacttcagagagggtgatatgatcgcagtgcctactggtgttgcatggtggatgtggaacaatgaagacactcctgttgttgccgtttctattattgacaccaacagcttggagaaccagatggaccagatgcctaggagattctatcttgctgggaaccaagagcaagagtttatgaaatatatgatgatgatgggaggtcatatgagccagaaaggaaagcatcagcaagaagaagaaaacgaaggaggcagcatattgagtggcttcaccatggaattcttggaacatgcattcagcgtggacaagcagatagcgaaaaacctacaaggagagaacgaaggggaagacaagggagccattgtgacagtgaaaggaggtatgagcgtgataaaaccacccacggacgagcagcaacaaagaccccaggaagaggaagaagaagaagaggatgagaagccacagtgcaagggtaaagacaaacactgccaacgcatgatgatgatgatgatgatgcgaggaagccaaagcaaaagcagaagaaatggcattgacgagaccatatgcaccatgagaatgcgccacaacattggccagacttcatcacctgacatctggaaccctcaagccggtagcgtcacaaccgccaccagcatggacttcccagccctctcgtggctcagactcagtgctgagtggggatctctccgcaagaatgcaatgttcgtgccacactacaacatgaacgcgaacagcataatatacgcaatgaatggacgggcaatgatacaagtggtgaattgcaacggtgagagagtgtttgatggagagctgcaagagggacgggtgctgatcgtgccacaaaactttgtggtggctgcaagatcacagagtgacaacttcgagtgggtgtcattcaagaccaatgatacacccatgatcggcactcttgcaggggcaaactcattgttgaacgcattaccagaggaagtgattcagcacactttcaacctaaaaagccagcaggccaggcagataaagaacaacaacccttggaagttcctggttccacctatgatgatgatgatgatgcaggagtctcagaagagagctgtggcttag

SEQ ID NO: 22 - DNA, 20 bp, Glycine max
gatgccgtgaaacgccgcgc

SEQ ID NO: 23 - DNA, 20 bp, Glycine max
gccgccgcggcagacgagaa

SEQ ID NO: 24 - DNA, 20 bp, Glycine max
gctgccgcggccgccgagaa

SEQ ID NO: 25 - DNA, 20 bp, Glycine max
gatacgtggatatattattg

SEQ ID NO: 26 - DNA, 20 bp, Glycine max
gaattaagcgatgaagatca

SEQ ID NO: 27 - DNA, 20 bp, Glycine max
gttgtctctatgttattgtt

SEQ ID NO: 28 - DNA, 20 bp, Glycine max
gttgttgtgtagcaatccga

SEQ ID NO: 29 - DNA, 1347 bp, Glycine max
atggcttcctcaatgatctcctccccagctgttaccaccgtcaaccgtgccggtgccggcatggttgctccattcaccggcctcaaaagcatggctggcttccccacgaggaagaccaacaatgacattacctccattgctagcaacggtggaagagtacaatgctttttgaaatccgatgcaagcaaaacgattcatgccgctgaaagactgggtaggggtattgagactgatggaattaccacccctgtggttaacacttctgcctacttttttaagaaaaccgctgatctcattgatttcaaggagaatcgtcaagtgagttatgaatacgggcgctatggaaacccaacgacggtggttctggaggagaagataagtgcattggagggggccgaatcaactgtgataatggcgtctgggatgtgtgctagcgtagtcctgtttatggcactggttccagctggtggacatcttgtgaccactacggattgttataggaagactagaatattcattgagacttttcttccaaagatggggatcacgaccactgtaattgatccagcagatgttggagccttggaatctgcattggagcagcacaatgtgtctctattcttcactgagtctcctaccaatccattcctgagatgtgttgatattaagctggtttcagagctttgccacaagaaggggactttgctctgtattgatggtacatttgcaactccattgaaccagaaggcccttgcccttggcgctgatctgattctgcactccttaacaaaatacatgggtggacatcatgatgtccttggtggttgcataagtggttcaattaaggtggtttcgcaaattcggactttgcaccatgttttgggtggtacacttaacccgaatgctgcatacctattcatcagaggcatgaaaacgctgcatctccgtgtacagcagcagaattcaacaggaatgaggatggccaaacttttagaggcacatcccaaggtgaagcgggtctactatccaggcttgccgagtcaccctgaacatgagcttgccaagaggcagatgactggtttcggtggtgttgtcagttttgagattgatggagatctacataccacaataaaatttattgattcattgaaaatcccatatattgcggcctcgtttggtggctgtgagagcattgtggatcaacctgctattttgtcttactgggatcttcctcagtcagaaagggccaagtacaagatttatgacaacctggttcgcttcagctttggagttgaagattttgaggatttgaaggctgatgtcctgcaagctctggaagctatatag

SEQ ID NO: 30 - DNA, 751 bp, Glycine max
gagctctttttctgccacacttcaattcaaaggccatagtggtactagtgattaatgaaggagaagcaaacattgaacttgttggcattaaagaacaacaacagaggcagcaacaggaagagcaacctttggaagtgcggaaatatagagctgaattgtctgaacaagatatatttgtaatcccagcaggttatccagttgtggtcaacgctacctcagatctgaatttctttgcttttggtatcaatgccgagaacaaccagaggaacttccttgcaggtgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcagacctgcaaggaagttcctctggttgttctcggcattgataccaaaagcaaagaaattcagatctgaggtagcgttgaccacaactggataacctgctgggattacaaatatatcttgttcagacaattcagctctatatttccgcacttccaaaggttgctcttcctgttgctgcctctgttgttgttctttaatgccaacaagttcaatgtttgcttctccttcattaatcactagtaccactatggcctttgaattgaagtgtggcagaaaaagagctc

SEQ ID NO: 31 - DNA, 466 bp, Glycine max
atggccgtttcgagctcgcacatgcgtttcacctttgagtgccgctccgatcccgatttctcgccccccccgccgtccttcgacaacctccgccgccgaaacttccgctcctccgcaggatccggcgcggcgtttcacggcatctcctccctcatcctccgcttccctcccaacttccagcgccagctaagcaccaaggcgcgccgcaactgcagcaacatcggcgtcgcgcaaatcgtcgccgcttcgtggtcgaacaacagcgacaactctccggccgccggggctccggcgccgcccgcggccaccgccacggacgccgctacggtgcctctccccgtcgtcgtcgccgccaacgaggacgtcgttgtctccgccgcggcagacgagaacggggctgtacagttaaacagtagttcttattcttcatttttgaaatccgatgcaagcaaaacgattcatgccg

SEQ ID NO: 32 - DNA, 463 bp, Glycine max
atggccgtttcgagctcgcacatgcgtttcaccttcgagtgccgctccgatcccgatttctcgccaccgccgccgtccttcgacaacctccgccgccgtaacttccgctcctccgccggatccggcgcggcgtttcacggcatctcctccctcatcctccgattccctcccaacttccagcgccagctcagcaccaaggcgcgccgcaattgtagcaacatcggcgtcgcgcaaattgtcgccgcttcgtggtcgaacaacagcgacaactctccggccgccggggctccggcgccgcccgcggccacagcagcggacgccgccacggtgcctctccccgtcgacctcgccgccgccgaggacgtcgtcgtctctgccgcggccgccgagaacgggactgtacagttaaacagtagttcttattcatttttgaaatccgatgcaagcaaaacgattcatgccg

SEQ ID NO: 33 - protein, 155 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATATDAATVPLPVVVAANEDVVVSAAADENGAVQLNSSSYSSFLKSDASKTIHA

SEQ ID NO: 34 - protein, 154 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATAADAATVPLPVDLAAAEDVVVSAAAAENGTVQLNSSSYSFLKSDASKTIHA

SEQ ID NO: 35 - protein, 68 aa, Glycine max
MAVSSSHMRFTFECRSDPDFSPPPPSFDNLRRRNFRSSAGSGENGAVQLNSSSYSSFLKSDASKTIHA

SEQ ID NO: 36 - protein, 67 aa, Glycine max
MAVSSSHMRFTFECRSDPDFSPPPPSFDNLRRRNFRSSAGSGENGTVQLNSSSYSFLKSDASKTIHA

SEQ ID NO: 37 - protein, 62 aa, Glycine max
MAVSSSHMRFTFECRSDPDFSPPPPSFDNLRRRNFRSSAGSVQLNSSSYSFLKSDASKTIHA

SEQ ID NO: 38 - DNA, 20 bp, Glycine max
ggccgcaactgcagcaacat

SEQ ID NO: 39 - DNA, 20 bp, Glycine max
ggccgcaattgtagcaacat

SEQ ID NO: 40 - DNA, 20 bp, Glycine max
ggcaacgagcgtggcatgct

SEQ ID NO: 41 - DNA, 20 bp, Glycine max
gtcagtttgtgtctcattaa

SEQ ID NO: 42 - DNA, 20 bp, Glycine max
gtcagtttctgtctcattac

SEQ ID NO: 43 - protein, 536 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATATDAATVPLPVVVAANEDVVVSAAADENGAVQLNSSSYSSFLKSDASKTIHAAERLGRGIETDGITTPVVNTSAYFFKKTADLIDFKENRQVSYEYGRYGNPTTVVLEEKISALEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDVLGGCISGSIKVVSQIRTLHHVLGGTLNPNAAYLFIRGMKTLHLRVQQQNSTGMRMAKLLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 44 - protein, 535 aa, Glycine max
MAVSSSHMRFTFECRSDPDFSPPPPSFDNLRRRNFRSSAGSGAAFHGISSLILRFPPNFQRQLSTKARRNCSNIGVAQIVAASWSNNSDNSPAAGAPAPPAATAADAATVPLPVDLAAAEDVVVSAAAAENGTVQLNSSSYSFLKSDASKTIHAAERLGRGIETDGITTPVLNTSAYFFKKTADLIDFKENRQVSYEYGRYGNPTTVVLEEKISILEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGGHHDALGGCISGSTKVVSQIRTLHHVLGGTLNPNAAYLFIRGMKTLHLRVQQQNSTGMRMAELLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 45 - DNA, 751 bp, Glycine max
atggctgaaagcgcggtggtgaacaggaagcgcagcggcacggacgacgtcgtttcggagaacgacggaaagaggaagaatgtggtggcacccgacatggaccctgcggcggcattggccttgacgcggcacgagttcggggagcacggtggcgtgaacatgtcgatcgaggcctcggctacgttcacggtgatggagccggagaccctctgccgcatgttcactggcgagctgggccccgaccgcgacttcttcatctacagccgccacttcaacccgacggtgctcaacttgggccgccacatggccgccctggaaggcacggaggccgcctactgcaccgccagcggcatggccgccatctcctcggtcctcctccagctctgcagctccggcgggcacgtggtggcctccaccactctctacggcgggacccacgccctcctcacgcacttcctccccgggaaatgcaacattagcaccaccttcgtggacatcagtgatttggaaagcgtggagagtgcgatcgtggaagggaagacgaacgtcctgtacttcgagtccatggcgaatccgagcctgaaggtcgcaaacattccggaactgtgccgcgtgggacacgcaaagggcgtcacagttgtcaccgacaacaccttcgctcccatggtcatctcccccgctcgtctcggcgctgacgtcgtcgttcacagcatctccaagtttatcagtgggggtgctgatattattgcag

SEQ ID NO: 46 - DNA, 751 bp, Glycine max
atggctgaaagcgcggtgacgaacaggaagcgcagcagcggggacgacgccgtttcggagaatgacagaaagcggaagaatatggtggcacgcaacatggacccggctgaggcgttggccttgacgcggcacgagttcggggagcacggtggcgtaaacatgtcgatcgaggcctcggcgacgttcacggtgatggagccggagaccctcggtcgcatgttcgccggcgagctgggcccggaccgcgatttcttcatttacagccgccacttcaacccgacggttgtcaacctgggccgccacatggccgccctggaaggcacggaggccgcctactgcacagccagcggcatggccgcgatctcctcggtactcctccagctctgtagcaccggcgggcacgtggtcgcctccaccactctctatggtgggactcacgccctcctcacgcacttcctccccgggaaatgcaacatcaccacgacgttcgtggacatcgccgatttggaaagcgtggagagtgccatcgtggaagggaagacgaacgtgctgtacttcgagtccatggcgaatccgagcctgaaggtcgcagacattccggaactgtgccgcgtgggacacgcaaagggcgtcacggtggtcgtcgacaacaccttcgctcccatggtcatctcccccgctcgtctcggcgctgacgtcgtcgttcacagcatctcaaagtttatcagtggcggtgctgatattattgcag

SEQ ID NO: 47 - DNA, 20 bp, Glycine max
gcccgacatggaccctgcgg

SEQ ID NO: 48 - DNA, 20 bp, Glycine max
gcgcaacatggacccggctg

SEQ ID NO: 49 - DNA, 20 bp, Glycine max
ggatcccattgtttaggttc

SEQ ID NO: 50 - DNA, 20 bp, Glycine max
gttgtgctttagttctaccc

SEQ ID NO: 51 - DNA, 20 bp, Glycine max
gagcttatcccgtcctattg

SEQ ID NO: 52 - DNA, 20 bp, Glycine max
gttgctaaccgatggattag

SEQ ID NO: 53 - protein, 440 aa, Glycine max
MAESAVTNRKRSSGDDAVSENDRKRKNMVARNMDPAEALALTRHEFGEHGGVNMSIEASATFTVMEPETLGRMFAGELGPDRDFFIYSRHFNPTVVNLGRHMAALEGTEAAYCTASGMAAISSVLLQLCSTGGHVVASTTLYGGTHALLTHFLPGKCNITTTFVDIADLESVESAIVEGKTNVLYFESMANPSLKVADIPELCRVGHAKGVTVVVDNTFAPMVISPARLGADVVVHSISKFISGGADIIAGAVCGPASLVNSMMDLHDGSLMLLGPTMNAKVAFELSERIPHLSLRMKEHCHRALVFATRLKKLGLKVIYPGLEDHPQHKLLKSIANREYGYGGLLCIDMGTVERANLLMAHLQNDAQFGFMAVSLGYYETLMSCSGSSTSSEMTEEEQKLAGISPGLVRMSIGYIGTLEQKWSQMEMALERFQEKEPLV

SEQ ID NO: 54 - protein, 440 aa, Glycine max
MAESAVVNRKRSGTDDVVSENDGKRKNVVAPDMDPAAALALTRHEFGEHGGVNMSIEASATFTVMEPETLCRMFTGELGPDRDFFIYSRHFNPTVLNLGRHMAALEGTEAAYCTASGMAAISSVLLQLCSSGGHVVASTTLYGGTHALLTHFLPGKCNISTTFVDISDLESVESAIVEGKTNVLYFESMANPSLKVANIPELCRVGHAKGVTVVTDNTFAPMVISPARLGADVVVHSISKFISGGADIIAGAVCGPASLVNSMMDLHDGSLMLLGPTMNAKVAFELSERIPHLSLRMKEHCHRALVFATRLKKLGLKVIYPGLEDHPQHELLKSIANREYGYGGLLCIDMETVERANLLMAHLQNDAQFGFMAVSLGYYETLMSCSGSSTSSGMTEEEQKLAGISPGLVRMSIGYIGTLEQKWSQMEIALDRFQEKQPLV

SEQ ID NO: 55 - protein, 855 aa, Glycine max
MLGNGVVGILAESVNKWERRAPLTPSHCARLLHGGTGVSRIIVQPSTKRIHHDALYEEVGAEISQDLSQCGLILGIKQPKLEMILPDRAYAFFSHTHKAQKENMPLLDKILAERASLYDYELIVGDNGKRLLAFGKFAGRAGMIDFLRGLGQRFLSLGYSTPFLSLGSSYMYPSLAAAKAAVISVGEEIATQGLPLGICPLVFVFTGSGNVCSGAQEIFKLLPHTFVDPSKLRDLHRTDKDQPRHASKRVFQVYGCVVTAQDMVEPKDHVIVFDKADYYSHPEHYNPTFHEKIAPYASVIVNCMYWEKRFPQLLSYKQMQDLMSQGCPLVGIADITCDIGGSIEFVNRATSIDSPFFRYDPLTNSYHDDMEGNGVICLAVDILPTEFAKEASQHFGNILSQFVINLASATDITKLPAHLRRACIANKGVLTSLYDYIPRMRSSDSEEVSENAENSLSNKRKYNISVSLSGHLFDQFLINEALDIIEAAGGSFHLVNCHVGQSVEAVSFSELEVGADDRAVLDQIIDSLTAIANPTENDRFSNQDSSKISLKLGKVEENGIEKESDPRKKAAVLILGAGRVCQPAAEMLSSFGRPSSSQWYKTLLEDDFECQIDIEIIVGSLYLKDAEQTVEGIPNVTGVQLDVMDHANLCKYIAQVNVVISLLPPSCHIIVANACIELKKHLVTASYVDSSMSMLNDKAKDAGITILGEMGLDPGIDHMMAMKMINQAHVRKGKIKSFTSYCGGLPSPEAANNPLAYKFSWNPAGAIRAGRNPATYKWGGETVHIDGNDLYDSATRLRLPDLPAFALECLPNRNSLLLRDLYGITEASTIFRGTLRYEALDIIEAYGIKLIETTE

SEQ ID NO: 56 - protein, 1048 aa, Glycine max
MLGNGVVGILAESVNKWERRAPLTPSHCARLLHGGTGVSRIIVQPSTKRIHHDALYEEVGAEISQDLSQCGLILGIKQPKLEMILPDRAYAFFSHTHKAQKENMPLLDKILAERASLYDYELIVGDTGKRLLAFGKFAGRAGMIDFLRGLGQRFLSLGYSTPFLSLGSSYMYPSLAAAKAAVISVGEEIATQGLPLGICPLVCLFTGSGNVCSGAQEIFKLLPHTFVDPSKLRDLHRTDPDQPRHASKRVFQVYGCVVTAQDMVEPKDPVKVFDKADYYAHPEHYNPTFHEKIAPYASVIVNCMYWEKRFPQLLSYKQMQDLMGRGSPLVGIADITCDIGGSIEFVNRSTSIDSPFFRYDPLTNSYHDDMEGNGVICLAVDILPTEFAKEASQHFGNILSQFVVNLASATDITKLPAHLRRACIAHKGVLTSLYDYIPRMRSSDSEEVSENSENSLSNKRKYNISVSLSGHLFDQFLINEALDIIEAAGGSFHLVNCHVGQSIEAVSFSELEVGADNRAVLDQIIDSLTAIANPTEHDRFSNQDSSKISLKLGKVEENGIEKESDPRKKAAVLILGAGRVCQPAAEMLSSFGRPSSSQWYKTLLEDDFECQTDVEVIVGSLYLKDAEQTVEGIPNVTGIQLDVMDRANLCKYISQVDVVISLLPPSCHIIVANACIELKKHLVTASYVDSSMSMLNDKAKDAGITILGEMGLDPGIDHMMAMKMINQAHVRKGKIKSFTSYCGGLPSPEAANNPLAYKFSWNPAGAIRAGRNPATYKWGGETVHIDGDDLYDSATRLRLPDLPAFALECLPNRNSLLYGDLYGITEASTIFRGTLRYEGFSEIMGTLSRISLFNNEAHSLLMNGQRPTFRKFLFELLKVVGDNPDELLIGENDIMEQILIQGHCKDQRTAMETAKTIIFLGLLDQTEIPASCKSAFDVACFRMEERLSYTSTEKDMVLLHHEVEIEYPDSQITEKHRATLLEFGKTLDEKTTTAMALTVGIPAAVGALLLLTNKIQTRGVLRPIEPEVYNPALDIIEAYGIKLIEKTE

SEQ ID NO: 57 - protein, 439 aa, Glycine max
MMRVRFPLLVLLGTVFLASVCVSLKVREDENNPFYLRSSNSFQTLFENQNGRIRLLQRFNKRSPQLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLLGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELVGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 58 - protein, 542 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERKQEEDEDEEQQRESEESEDSELRRHKNKNPFLFGSNRFETLFKNQYGRIRVLQRFNQRSPQLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGI

SEQ ID NO: 59 - protein, 605 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERKQEEDEDEEQQRESEESEDSELRRHKNKNPFLFGSNRFETLFKNQYGRIRVLQRFNQRSPQLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 60 - protein, 439 aa, Glycine max
MMRVRFPLLVLLGTVFLASVCVSLKVREDENNPFYLRSSNSFQTLFENQNGRIRLLQRFNKRSPQLENLRDYRIVQFQSKPNTILLPHHADADFLLFVLSGRAILTLVNNDDRDSYNLHPGDAQRIPAGTTYYLVNPHDHQNLKIIKLAIPVNKPGRYDDFFLSSTQAQQSYLQGFSHNILETSFHSEFEEINRVLFGEEEEQRQQEGVIVELSKEQIRQLSRRAKSSSRKTISSEDEPFNLRSRNPIYSNNFGKFFEITPEKNPQLRDLDIFLSSVDINEGALLLPHFNSKAIVILVINEGDANIELVGIKEQQQKQKQEEEPLEVQRYRAELSEDDVFVIPAAYPFVVNATSNLNFLAFGINAENNQRNFLAGEKDNVVRQIERQVQELAFPGSAQDVERLLKKQRESYFVDAQPQQKEEGSKGRKGPFPSILGALY

SEQ ID NO: 61 - protein, 621 aa, Glycine max
MMRARFPLLLLGVVFLASVSVSFGIAYWEKQNPSHNKCLRSCNSEKDSYRNQACHARCNLLKVEEEEECEEGQIPRPRPQHPERERQQHGEKEEDEGEQPRPFPFPRPRQPHQEEEHEQKEEHEWHRKEEKHGGKGSEEEQDEREHPRPHQPHQKEEEKHEWQHKQEKHQGKESEEEEEDQDEDEEQDKESQESEGSESQREPRRHKNKNPFHFNSKRFQTLFKNQYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 62 - protein, 596 aa, Glycine max
MIARFPLLPLLLLGIVFLASVSVSLQVKPPSHHECLQSCESETDPYKRKACKLSCKFVPEHGEQQHEEEEREREHPQPHPPHEERGRRERGRQEGEKEEKEQDERELQPRPFPRPEEHEGEQWKGRHRHEDPEERARMRLREAERIEREREEQRVREEEEKLRRREKKEQREEKEEEGQGSEDSHSKRQNNPFHFSSNRFQTLFKNQHGHLRVLQRFDQRSPQLENLRDYRVVELMAKPNTLFLPHHADADFLLLVLSGRALINLVKPDDRDPYYLDRGYAQRIPAGTTVYLVNPDKKKDLRVIKLAIPVNKPGNFEDFFLSSTQDQQSYLQGFSENILEASFNTKFEEINRVLFGGEGRRHQQEGVILELSKEQIRELSKRAKSSSRSTNSFDYEPFYLRGSQISSNNFGKFYEITPEKNPQLRDFDILLNTVDINELVILIIETKQIQGGLLLPHYNSKAIVILMVTEGEANIELVGLKEQQQGEETREVRKYRAELSEDDIFVIPAAYPFVVNATSNLNFVAFGINAENNQRNFLAGSKDNVIRQIQKQVKELAFPAGSAQDIENLIKNQRESYFADAQPLQKEEGKKGSFVF

SEQ ID NO: 63 - protein, 353 aa, Glycine max
MRVRRRKRNKTTVSNLVRFHVLITNSTNSTNGLMEILKNVKVRMSQSQKRNNLYHFSSNSFDTLFKNQYGHIRLFQRFNQRSPQLENLRDYHVVEFKFNPNALFLPHHADSEFLLERLHPGDALRIPAGTTYYLVNPDYKETFPIVKIINLCLFRVEFLPIQQSYLQGFSENILEASFNTEFEEINRVLLGEEGQQQGVQRLQERVMVELSKEQIQELSRHAKSSSRKTITSKNEPFNLRSRKPIYSNKFGALLLPHFNSKAIVILVINEGEANIELQLIHLSSNLNFLAFGINAENNQRNFPAEGAVPADVLGLAPALAADHRNLREVDGGAAPLAAEDVLECRERRSVEKK

SEQ ID NO: 64 - protein, 45 aa, Glycine max
HGISSLILRFPPNFQRQLSTKARRNCSNIGVAQIVAAWSNNSDNS

SEQ ID NO: 65 - DNA, 20 bp, Glycine max
gaactcccaaccgttaaact

SEQ ID NO: 66 - DNA, 20 bp, Glycine max
gcactctggatcaacttatt

SEQ ID NO: 67 - DNA, 20 bp, Glycine max
gtatatcgatagctgtgtta

SEQ ID NO: 68 - DNA, 20 bp, Glycine max
gtcaatgtcttctccataag

SEQ ID NO: 69 - DNA, 20 bp, Glycine max
gctgtggaactaaattcatt

SEQ ID NO: 70 - DNA, 20 bp, Glycine max
gtcctaagggtgccacgtgg

SEQ ID NO: 71 - DNA, 20 bp, Glycine max
gctaaccgccaagcaagaag